(19) United States
(12) Patent Application Publication
(10) Pub. No.: US 2008/0065418 A1
(43) Pub. Date: Mar. 13, 2008

(54) Systems and Methods to Manage Drug Accountability Processes in Clinical Trials

(76) Inventors: William Douglas Byrom, Nottingham (GB); Nicola Frances Petrina Dowlman, Nottingham (GB); Michelle Mijung Kwak, Nottingham (GB); Marie MacDonald, Buffalo Grove, IL (US); Graham John Nicholls, Nottingham (GB); Robyn Ian Wood, Mid Glamorgan (GB)

Correspondence Address: CASTELLANO PLLC, P.O. Box 1555, Great Falls, VA, 22066, US

(21) Appl. No.: 11/530,167

(22) Filed: Sep. 8, 2006

(51) Int. Cl. G06F 19/00; A61B 5/00

(52) U.S. Cl. 705/3; 600/300

(57) ABSTRACT

Systems and methods for managing drug accountability activities wherein various stakeholders such as Sponsors, monitors, site investigator personnel, and depot personnel are provided with access to data related to a clinical trial for the management of accountability, returns, reconciliation, and destruction processes. The systems and methods includes reporting, query, data compilation and parsing, commenting, tracking, and other features to help manage drug accountability processes for clinical trials.

### BACKGROUND OF THE INVENTION

1. Field of the Invention

The present invention generally pertains to software, systems, and methods for managing clinical trials, and more particularly, managing drug supply processes during clinical trials using software and computer system architectures.

2. Background Information

Clinical trials are required studies for regulatory approval of candidate drugs. The importance and scope of clinical trials have lead to the proliferation of management systems and methods to help administer many aspects of the clinical trial process. For example, the FDA requires that new drugs undergo clinical trials to test the safety, efficacy and other effects of new drugs. The investigational drug is evaluated through the observation of subjects who are either provided the drug or a comparative, which may be a placebo.

Typically, development clinical studies are conducted in three phases, Phase I, Phase II, and Phase III. Additionally, clinical trials may be conducted on marketed drugs such as Phase IV studies, Investigator-led studies and post-marketing surveillance studies. Phase I clinical trials typically involve a small number of subjects and are carried out with the aim of characterizing the perforance of the drug, and more particularly determining the safety of the drug in subjects. A phase I trial will often study the effects of the drug on healthy subjects and include various text parameters such as absorption efficacy, metabolization rate, side effects, and other safety parameters.

Upon successful completion of Phase I clinical trials (i.e., the determination that the new candidate drug is provisionally safe), a Sponsor may initiate a Phase II clinical trial. Phase II clinical trials usually involve a larger subject pool and are designed to determine the optimal dose of the candidate drug with respect to efficacy and safety. Phase II trials are often controlled studies in that a number of subjects may be given a placebo as a control group. Furthermore, Phase II trials are targeted to treat patients suffering from the target disease indication. Thus the efficacy and safety of various dose levels of the candidate drug under investigation may be studied in a controlled group.

When the results of a Phase II clinical trial identify the optimal dose of the candidate drug to treat a medical condition, Phase III trials are commenced. Phase III clinical trials are designed to determine the safety and efficacy of the candidate drug over a large population. Accordingly, Phase III trials often involve hundreds to thousands of subjects that may be spread over a diverse geographical region. Additionally, Phase II trials may involve subjects from different ethnic, cultural, or genetic backgrounds.

Any number of different stakeholders may be involved in a clinical trial. Sponsors usually begin the clinical trial process and may be responsible for initiating the clinical trial, applying for regulatory approval, and recruit study sites and/or financing of the clinical trial. Sponsors are often the pharmaceutical or biotechnology company that discovered the candidate drug or may be an entity with a substantial financial or other interest in the candidate drug. The various tasks attendant to conducting and monitoring a clinical trial may be conducted by individuals or groups of individuals within the Sponsor's organization. Alternatively, Sponsors may seek entities, firms, or other individuals to conduct or manage one or more aspects of the clinical trial.

In many instances, Sponsors utilize Contract Research Organizations (“CROs”) to help conduct clinical trials. Typically, CRO's are used to monitor clinical trials and are given the responsibility of ensuring compliance with various scientific and regulatory guidelines. Additionally, CRO's may be responsible for record keeping and other supply management duties. CRO's may employ various personnel to conduct one or more of the services required including, but not limited to, logistic support, data collection, monitoring, etc.

Regulatory requirements for clinical trials vary from country to country. Nonetheless, for practically all clinical trials it is necessary to perform drug accountability, reconciliation, returns and destruction (“ARRD”) activities. ARRD activities form part of the total drug supply chain management that is part of any clinical trial. More particularly, ARRD activities require full documentation of the chain of custody for study material. Clinical trial Sponsors are required to provide an audit trail for product (i.e. medication unit, treatment pack, packets, consignments, etc.) at all times during the lifecycle of the product.

For example, Good Manufacturing/Clinical and Distribution Practices mandate that drug ARRD procedures must be implemented in all investigational clinical trials. Both European and U.S. regulations dictate the type of information and management practices that must be considered at the inception of every study.

According to the European Commission's Good Manufacturing practices guide Annex 13:
The delivered, used and recovered quantities of product should be recorded, reconciled and verified by or on behalf of the sponsor for each trial site and each trial period. Destruction of unused investigational medicinal products should be carried out for a given trial site or given trial period only after any discrepancies have been investigated and satisfactorily explained and the reconciliation has been accepted. Recording of destruction operations should be carried out in such a manner that all operations may be accounted for. European Commission Enterprise Directorate-General, Annex 13: Manufacture of Investigational Medicinal Products, (EC, Brussels, July 2003).

FDA regulations also state that the investigator is responsible for the proper and secure physical storage and recordkeeping of investigational agents. More specifically the regulations state that the investigator must:
Maintain a careful record of the receipt, use and final disposition of all investigational agents received using the Drug Accountability Record Form;
Store the agent in a secure location, accessible to only authorized personnel, preferably in the pharmacy;
Maintain appropriate storage of the investigational agent to ensure the stability and integrity of the agent;
Return any unused investigational agents at the completion of the study or upon notification that an agent is being withdrawn. Food and Drug Administration, 21 C.F.R. 312 Investigational New Drug Application (FDA, Rockville, Md., April 2005); 21 C.F.R. 211 Current Good Manufacturing Practice for Furnished Pharmaceuticals (FDA, Rockville, Md., April 2003); 21 C.F.R. 210 Current Good Manufacturing Practice in Manufacturing, Processing, Packing, or Holding of Drugs (FDA, Rockville, Md., April 2005).

The intent of these drug accountability procedures is to ensure patient safety by assisting the investigator in making certain that the correct blinded drugs are used only for patients enrolled in an approved protocol.

Regulatory bodies conduct audits for various reasons but always with the same goal: to ensure the safety of research participants and to guarantee the accuracy the clinical data collected. Because these bodies define research procedures for study conduct and, ultimately, they grant approvals based upon the integrity of the data collected, agencies use inspections to ensure for themselves that the regulations are complied with, subject safety is safeguarded and data are collected and reported appropriately.

Clinical Investigators are well aware of the possible consequences of a negative regulatory inspection. At worst these might result in disqualification from research and possible jail sentencing. More likely, disciplinary actions range from a figurative slap on the wrist to restrictions that limit participation in future projects. However, proper drug accountability procedures and regular checks during monitoring visits could have prevented the majority of the drug accountability findings noted in recent FDA warning letters. There are currently many warning letters specifically mentioning drug accountability violations on the FDA's website. These letters include warnings on aspects such as incomplete or inaccurate dispensing records, failure to maintain adequate drug inventory and the unavailability of drug distribution records.

Accordingly, the fundamental goal of any drug accountability system or method is to know at all times the exact status and location of all medications throughout the entire supply chain. The drug accountability process should effectively begin with the manufacture of the study drug. This process then follows the drug's entire lifecycle through packaging, shipment, dispensation, return, reconciliation and, ultimately, destruction. Existing processes at manufacturing sites mean that Sponsors tend to manage the production and packaging of supplies as this is within their direct control. However, once the drugs are shipped the process becomes much more difficult to manage, as information starts to appear in a number of formats from different sources.

In general investigative sites maintain two key types of documents relating to medication accountability: “master supplies/inventory documents” which detail shipments of medication received, current and historic inventory and medication returned to the depot, and a “subject dispensation documents” which record individual medication given to and returned by each patient or test subject. These two document types are typically the kep informational repositories of site-based drug accountability, although additional Sponsor and/or site specific documentation may also be used. Subject returns may be captured simply as the return of the pack dispensed, or more frequently, by capturing the quantity of medication returned.

The next step of drug reconciliation is typically performed by a monitor who verifies and reconciles all of the received, utilized and recovered medication for each site according to the Sponsor's written procedures. This entails checking the site documentation and physically verifying quantities and status of all relevant materials. The monitor then often coordinates the return of drugs from the site to an appropriate destination which, in most cases, is a local depot. The monitor may also identify and consolidate other supplies at the site that are no longer useable or necessary, such as damaged or expired medication.

Next, return shipments are sent to a designated depot accompanied by a document detailing quantities, individual contents and format of medication. A unique identifier is assigned to each shipment, and very often this is the only tracking device to link the shipment received at the depot with the individual medication packs sent from the site.

Upon receipt of a returns consignment, the depot must clearly identify the supplies, store them in an appropriately controlled area and maintain inventory records. In approximately 20% of cases, the depot is required by Sponsors to undertake a further reconciliation activity verifying received contents matched against those shown in the shipment documentation.

Finally, the returned materials must be destroyed. Returned supplies are generally stored at the depot until enough have accumulated to warrant sending a large shipment to the final destruction facility. At destruction, a certificate must be issued, and the study Sponsor must link the destruction information back to the individual medication units to prove both the identities and quantities of destroyed material.

Throughout the entire process, if any discrepancies are identified they must be investigated, explained and resolved.

Traditional ARRD methods have failed to address the complex and burdensome aspect of ARRD activities. While many clinical processes are progressing into the electronic age in an effort to make them faster, more efficient and more accurate, the ARRD process has lagged behind. Most clinical Sponsors address the ARRD regulations by relying heavily on inefficient manual paper-based systems with information coming from a number of sources in a variety of formats including, for example, electronic shipment files, emails, paper forms and hand written notes. This paper-based process makes it difficult to access information detailing the complete chain of custody for individual units of medication through a trial's lifecycle. Aggregate-level, particularly study or batch-level, transparency frequency is very poor, making it extremely difficult to obtain a holistic snapshot of the overall supply status. Mistakes are currently common due to multiple locations and formats of the same data, leaving studies vulnerable to audit enquiries. Furthermore, integration of ARRD activities with existing clinical trial management systems (CTMSs) and drug supply management systems (DSMSs) are lacking in functionality and compatibility.

Accordingly, a need exists for systems and methods for the efficient and accurate management of ARRD activities attendant to clinical trials.

### SUMMARY OF THE INVENTION

The system and methods disclosed herein pertain to managing ARRD activities. In one embodiment, a system is provided for managing ARRD activities that has a main database of information concerning drug accountability data for a clinical trial, a main processor controlling access to said main database, and at least one user processor in communication with said main processor to negotiate access to said main database. The system is running a software that allows the user to receive, enter, and transmit data concerning drug accountability processes for one or more clinical trials to one or more users.

In another embodiment, a method for managing drug accountability processes is provided. The method includes enabling an administrator to define a plurality of drug accountability parameters, storing the parameters in a central database, enabling a first user to enter drug accountability data corresponding to a clinical trial into the database and storing said data in the database, and enabling a second user to access the stored data. The users may also view, edit, parse, and compile the stored data.

In another embodiment, a method for managing drug accountability destruction processes is provided, the method including providing a system for retrieving viewing, storing, and entering treatment pack destruction activities. The method provides for tracking, managing, viewing, and reporting destruction activities related to consignments and mega-consignments.

In another embodiment, a system is provided that allows users to associate text entries, comments, queries, etc. with drug accountability data. The comments may then prompt other users of the system to take action, including actions relating to drug accountability processes, such as reconciliation processes or returns processes.

### BRIEF DESCRIPTION OF THE DRAWINGS

FIG. 1 is a diagram of the representative locations or individuals involved in the lifecycle of a candidate drug undergoing a clinical trial;

FIG. 2 is a diagram of representative locations or individuals involved in the lifecycle of a candidate drug undergoing a clinical trial;

FIG. 3 is a block diagram of the supply management and drug accountability processes encountered during a clinical trial;

FIG. 4 is a schematic representation of a computer system architecture of an embodiment of the present invention;

FIG. 5 is a block diagram of a process of the system and methods of the present invention;

FIG. 6 is a block diagram of processes of the system and methods of the present invention;

FIG. 7 is a block diagram of processes of the system and methods of the present invention;

FIG. 8 is a block diagram of processes of the system and methods of the present invention;

FIG. 9 is a block diagram of processes of the system and methods of the present invention;

FIG. 10 is a schematic of some of the functionalities and processes of the system and methods of the present invention;

FIG. 11 is a screenshot of a GUI employing some of the systems and methods of the present invention;

FIG. 12 is a screenshot of a GUI employing some of the systems and methods of the present invention;

FIG. 13 is a screenshot of a GUI employing some of the systems and methods of the present invention;

FIG. 14 is a screenshot of a GUI employing some of the systems and methods of the present invention;

FIG. 15 is a diagram illustrating the functionalities of an aspect of the system and methods of the present invention.

### DETAILED DESCRIPTION OF THE INVENTION

Systems and methods for effectively managing all aspects of ARRD activities in a clinical trial are described in detail herein. In the following description, numerous specific details are disclosed, such as various user architectures, user interfaces, methods, and workflows, to provide a through understanding of embodiments of the invention. One skilled in the relevant art will recognize, however, that the invention can be practiced without one or more of the specific details, or with other methods, components, etc. In other instances, well-known structures or operations are not shown or described in detail to avoid obscuring aspects of various embodiments of the invention.

Reference through this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places through this specification are not necessarily all referring to the same embodiment. Furthermore, the particular features, structures, or characteristics may be combined in any suitable manner in one or more embodiments.

As used herein, “candidate drug” means the drugs, compounds, substances, or mixtures that are being studied or is the subject of a clinical trial. Candidate drugs may refer to a combination of drugs or more than one drug, i.e. studies involving multiple drugs.

As used herein, “treatment drug” may refer to candidate drug, placebo, comparative drug, or any combination of the above that is provided to subjects of a clinical trial.

As used herein, “medication unit” refers to the individual unit of drugs that makes up the treatment drug dispensed to a patient or subject. The term medication may also include placebo or comparative drug. “Medication unit count” or “unit count” refers to the number of individual units of drug that are contained in any given treatment, pack, packet, or other grouping.

As used herein, “treatment unit” or “treatment packs” refers to a unit that is given to a subject at dispensation. The unit usually contains a number of medication units, i.e. pills, tablets, or other unit. Treatment units or treatment packs may be a pack, bottle, vial, medical device etc. Treatment packs may include more than one type of medication unit or treatment drug. Thus, for example, in a double-dummy study, a treatment pack may consist of one or more medication units of treatment drug in pill form and one or more medication units of placebo in tablet form. In the same study, a second treatment pack may consist of one or more medication units of treatment drug in tablet form and one or more medication units of placebo in pill form. As used herein, “pack” is used synonymously with treatment unit or treatment pack and generally refers to an individual treatment pack dispensed to a subject. Packs may be identified by a unique identifier for tracking, such as a unique number, barcode or RFID tag. Packs may also include labeling and instructions for the treatment drug being dispensed. Finally, packs may include comparative drug, which may for example be a drug that has been accepted by the medical community as a standard treatment for a particular treatment. Thus, treatment packs may include both candidate drug, comparative drug, placebo, and any combination of the aforementioned in a variety of different formats.

As used herein, “clinical trial” refers to any study in which a candidate drug is provided to a subject and data is collected regarding the effect of or absence of effect of the drug on a parameter of the study.

As used herein, “consignment” refers to the aggregation, collection, or other grouping of treatment packs. A return consignment or return refers to consignments shipped from a site to a depot. A supply consignment refers to consignments shipped from a depot to a site. A mega-consignment refers to the aggregation, collection, or grouping of consignments, typically for shipment from a depot to a destruction facility.

As used herein, “site” refers to the establishment or location from which a patient or subject is dispensed a candidate drug in a clinical trial. Examples of sites include, but are not limited to, hospitals, physician's offices, pharmacies, etc.

As used herein, “depots” or “distribution depots” refers to an establishment or location involved in the shipment and/or receipt of treatment drug during the product lifecycle. In some instances of a typical study chain, multiple depots may be used with in the supply cycle of the treatment drug. Thus, in this sense, a depot may ship treatment drug to a regional depot, which in turn ships treatment drug to a site.

As used herein, “destruction facilities” refers to an establishment or location at which returns are destroyed, typically via incineration. In some instances, a depot or site may also be a destruction facility.

As used herein, “clinical research organization” or “CRO” refers to an entity, firm, individual, organization, enterprise, or other company that coordinates and manages the execution of at least one aspect of a clinical study.

As used herein, “clinical research associate” or “CRA” or “monitor” refers to an individual who may be employed by a Sponsor or CRO to which certain clinical trial tasks are delegated. Monitors are typically responsible for various logistical processes to comply with regulatory and scientific guidelines. Moreover, CRAs may ensure that sites conduct clinical trials according to the clinical trial protocol.

As used herein, “Sponsor” refers to an entity, firm, individual, organization, enterprise, or other company that takes responsibility for the initiation and/or financing of the clinical trial. Typically, the Sponsor is a pharmaceutical or biotech company or the developer of the drug. Sponsors typically assume responsibility for applying to regulatory agencies for permission to conduct clinical trials, filing the results and progress reports during the clinical trial, and applying for regulatory approval at the conclusion of the program of clinical trials.

As used herein, “stakeholder” refers to an entity, firm, individual, organization, enterprise, or other company that has an interest in either a clinical trial or candidate drug. The interest may include financial, medical, or scientific interest. Stateholders may also be used herein synonymously with “user”. Accordingly a user of the system and methods described herein is also a stakeholder. Examples of the various stakeholders may include, but are not limited to, pharmaceutical companies, biotechnology companies, Sponsors, clinical research organizations, clinical research associates, trial managers, service support personnel, clinical investigator, study site coordinators, physicians, monitors, distribution depot personnel, destruction depot personnel, and nurse practitioners. Patients and/or subjects are not typically considered stakeholders or users. Similarly, only in rare circumstances are destructive depot personnel considered stakeholders.

As used herein, “Drug accountability, reconciliation, returns, and destruction” or “ARRD” refers to drug accountability, reconciliation, returns and destruction processes of a clinical trial. “Drug accountability” may be used generically to refer to ARRD activities. As used herein, “accountability” refers to determining the precise status, location, and contents of all treatment drug, treatment packs, and/or unit count in a clinical trial. Accountability includes information concerning the shipment receipt at a site, dispensation, and return by patient to a site of treatment drug, treatment packs, and/or unit count. As used herein, “reconciliation process” or “reconciliation” refers to the steps used to verify, authenticate, determine, or otherwise validate accountability by a stakeholder, usually a CRA/Monitor. As used herein, “returns process” refers to the steps used to log, store, modify data concerning, or otherwise record information relating to the return, receipt, or accounting of the treatment drug, treatment unit, or medication unit from a site to a depot, or a depot to a depot. Returns may also include undispensed treatment drug (e.g., superfluous, damaged, or expired units). Finally, as used herein, “destruction process” refers to the steps used to record, store, determine, or otherwise authenticate the destruction by a destruction facility of a candidate drug, medication unit, or pack.

As used herein, “supply management” processes refer to the steps used to track, record, store or otherwise view information concerning either the supply distribution or supply allocation of a drug.

As used herein, a “database management system” (“DBMS”) is a software program or set of programs that controls the organization, storage and retrieval of data (fields, records and files) in a database. It also controls the security and integrity of the database. DBMSs may provide users a number of different functions including a systematic approach to creating, maintaining, accessing, reporting, and analyzing data.

As used herein, “user privileges” refers to the association of a set of rules to a user such that a user is authorized with specific and/or controlled access to different functionalities of the system and methods.

As used herein, “rule set” or “rules” refers to logical instructions that form part of the software of the system such that upon the happening or non-happening of an event, a computing process occurs. In one aspect, rules may be used to define permission keys that either allow or prohibit access to a particular function of the system. Another example of a rule set is a time out operation. A time out operation is a rule where a user is logged off the system after a certain amount of inactivity has occurred. Rule sets may be singular rules or a combination of rules. Additionally, rule sets may result in different processing operations depending on the event, time, user, clinical trial, etc.

As used herein, “mobile computing platform” refers to a computer system that has at least a processor and a user interface for viewing data. A mobile computing platform may refer to a laptop, personal digital assistant, mobile phone, or other mobile computing systems. A mobile computing platform may contain a scanner or other device capable of detecting treatment packs, such as a barcode scanner or RFID scanner, integrated within the mobile computing platform or capable of being in communication with the platform.

As used herein, “parameters” refers to the delineation or defining of an aspect or part of ARRD activities into a discreet activity, function, or requirement. A parameter may include one aspect of an ARRD process. In other uses, parameter refers to the identification of a requirement or set of requirements as they relate to ARRD activities.

As used herein, “requirements” refers to the defining of a particular need, objective, or functionality of the system. A requirement may include a functionality as it relates to data related to ARRD activities, such as view pack, edit pack, or other functionality. A requirement may also refer to the ability of the system to provide a user with a certain ability related to ARRD activities or processes, such as add comment or run report.

As used herein, “schedule queue” refers to the list of schedulable items, which may be processed by the system. A schedulable item is a report or other compilation of data which can be sent, transmitted, or otherwise provided to a user. The system may provide said report or other compilation of data in any number of means including a text message, an outbound automated telephone call, attachment to an email, a fax or displayed on a web page.

As used herein, “alert” refers to the notification of a potential query, issue, notation, or other factor that is associated by the system to one or more users based on an event, such as, but not limited to, a new comment or other data entry event. An alert may be sent to a user electronically or otherwise reside or within the system for retrieval by a user.

### Typical Lifecycle of Drug

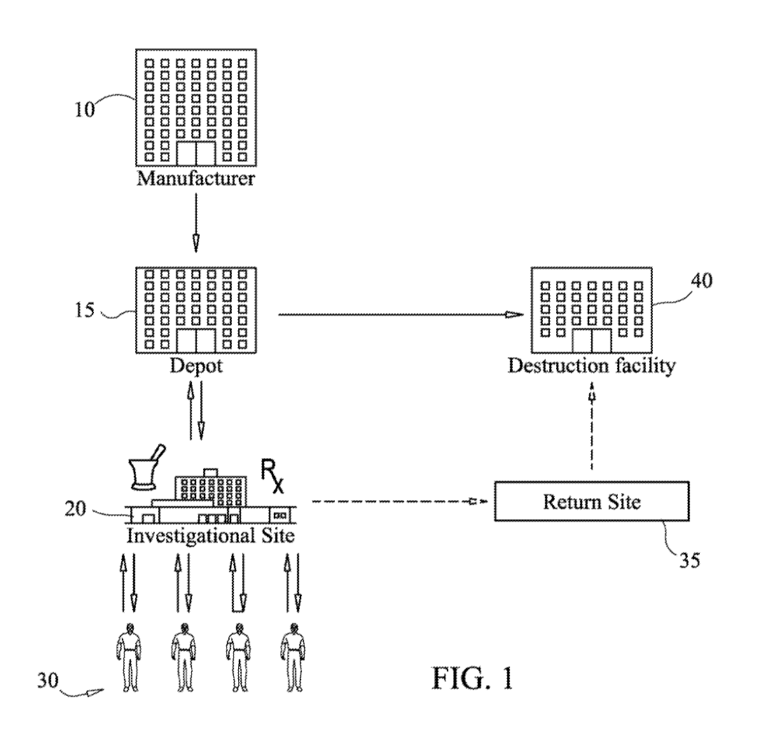

With reference to FIG. 1, the typical lifecycle of a candidate drug in a clinical trial begins at its manufacture and ends with its use or destruction. As seen in FIG. 1, a manufacturing site 10 ships treatment packs to distribution depots 15. Distribution depots 15 receives the treatments packs and may serve as regional distributors for treatment packs to various clinical trial or investigative sites 20. Each clinical trial site 20 may dispense treatment packs to one or more clinical trial patients or subjects 30. The lifecycle of the candidate drug continues, however, even after the drug has been dispensed to clinical trial subject 30. As discussed previously, test subjects 30 must return treatment packs to the clinical trial site 20 to ensure compliance with clinical study guidelines and regulations. Packs must be returned even if they contain no medication units. Receipt of the returned treatment packs must be accounted for. After receipt by the clinical trial site 20, the treatment packs may be grouped into a return consignment and returned to a depot 15 (or an alternatively designated return site 35), typically for shipment to a destruction depot 40. Alternatively, return consignments may be destroyed at the return depot 15 or site 20.

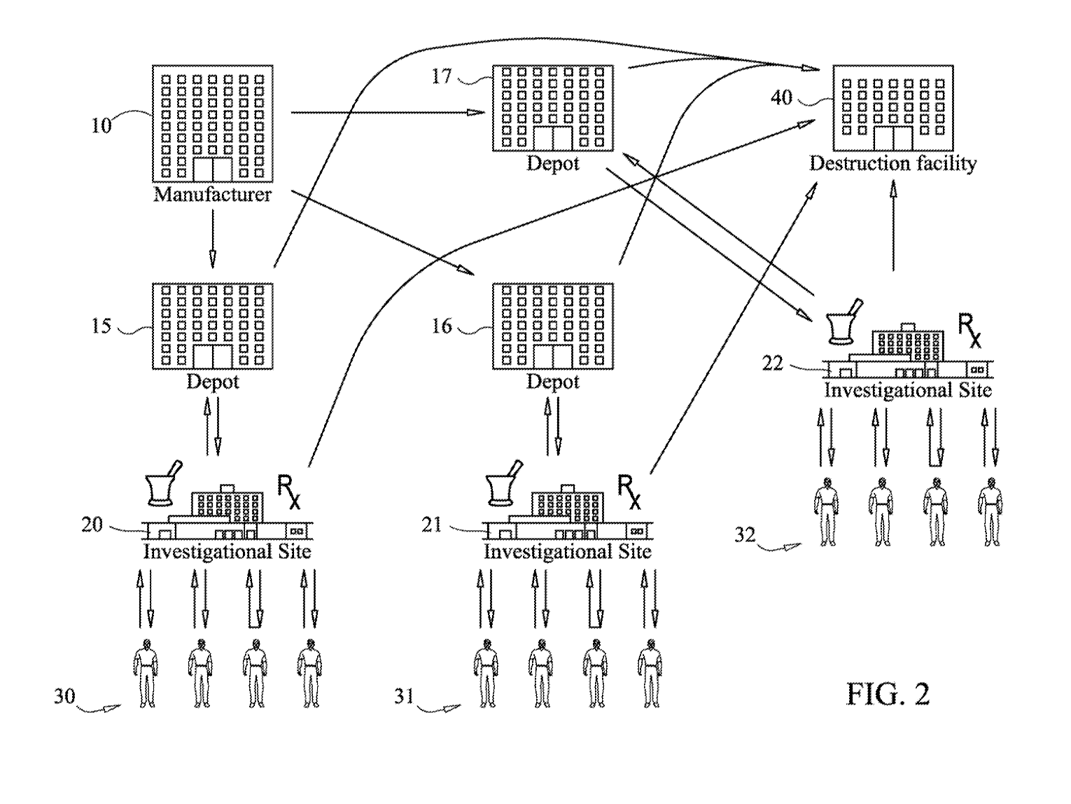

While only one manufacturer, depot, investigative site, and test subject are depicted in FIG. 1, it should be understood that for each clinical trial a number of different locations or subjects may be involved. For example, with reference to FIG. 2, a number of sites and stakeholders may be involved in a clinical trial during the lifecycle of the candidate drug and packs. As seen in FIG. 2, a manufacturing site 10 may ship candidate drug to several depots 15, 16, and 17. In turn, depots 15, 16, and 17 may ship treatment packs to a number of different investigative sites 20, 21, and 22, respectively. Finally, each investigative site 20, 21, and 22 may dispense treatment packs to numerous participants or subjects of a study, e.g. 30, 31, and 32. Moreover, treatment packs may then be returned to investigative sites 20, 21, 22, grouped into return consignments, and returned to depots 15, 16, and 17. The return consignments may then be shipped to one or more destruction facilities 40. In many instances, return consignments may be grouped into large mega-consignments at return depots 15, 16, and 17. The mega-consignment may then be shipped to a destruction facility 40 for destruction. As one of skill in the art would understand, alternative configurations may be involved in any particular clinical trials. Accordingly, the logistical workflow and audit trail of drug accountability can increase in complexity rapidly.

Typically any number of logistical activities must be accounted for during the lifecycle of a candidate drug and packs during a clinical trial. For example, logistical activities or parameters including, but not limited to, drug receipt at depot, drug receipt at site, subject randomization and pack allocation, subject dispensing and return documentation, review and confirmation of accountability data, management of return shipments, and destruction must all be recorded, stored, and made accessible to various stakeholders in the clinical trial process.

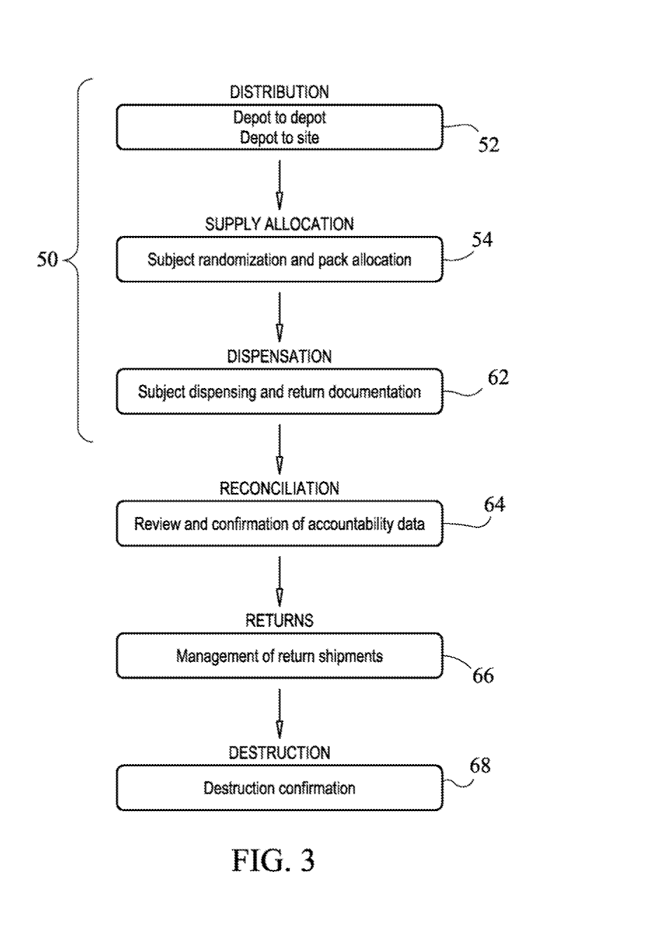

As seen in FIG. 3, during the lifecycle of a treatment drug, lifecycle processes can be categorized into component subparts, namely, accountability, reconciliation, returns, and destruction. As seen in FIG. 3, accountability processes 50 typically involve supply distribution processes 52, supply allocation processes 54, and dispensation processes 62. As one of skill in the art would understand, accountability processes 50 include supply distribution activities 52 such as the shipment and delivery of treatment drugs between depots and shipment from depots to sites. Supply allocation processes 54 refer to subject randomization and pack allocation in which treatment packs are allocated within the system to a specific patient. And dispensation processes 62 include on the one hand the physical dispensation of treatment packs to patients and the recordation of the dispensation; and on the other hand the receipt of returned medication packs and the recordation of the return and its contents.

Generally, existing supply and/or clinical trial management systems may perform or overlap with a few activities related to supply management processes. These activities may include for example limited tracking of distribution processes. For example, CTMS systems such as that disclosed in U.S. Patent Application Publication No. 2005/0055241 the entire contents of which are hereby incorporated by reference is a system that may perform some supply management processes. In addition, DSMS systems may manage ordering processes and shipment of treatment packs from manufacturing site to depots. Similarly, existing IVR providers may provide for the tracking of distribution and allocation data.

However, as seen in FIG. 3, full-scale drug accountability activities encompass a wide variety of activities and users. As discussed previously, accountability processes 50 includes processes such as distribution processes 52, allocation processes 54, and dispensation processes 62. Additional drug accountability processes include reconciliation processes 64, returns processes 66, and destruction processes 68. Reconciliation processes 64 include the verification of accountability by a CRA/Monitor. This process typically occurs at the study site. Return processes 66 include the collection, recordation and shipment of candidate drug, treatment packs, and/or medication units from a site to a depot. Returns may also include unused candidate drug, treatment packs, or unit counts that were not dispensed from a site to a patient. Finally, destruction processes 68 include verification of the destruction of a candidate drug, treatment unit, and/or medication unit. Typically, the destruction facility will provide a depot with a destruction certificate indicating that the received shipment has been destroyed. As can be seen in FIG. 3, the various logistical activities often mirror or coincide with the physical location of the candidate drug during its lifecycle. In other instances, however, users are often physically removed from the product lifecycle, making verification, reporting, data retrieval and other required activities difficult for some users. This is especially true where there are multiple stakeholders spread across a diverse region.

For example, while in the configuration described above some stakeholders are co-located with the product (i.e. depot manager—depot; investigator—hospital), often times stakeholders are not co-located with the product of interest. CRO personal, monitors, Sponsor management (i.e., trial manager), and others are often removed from the product lifecycle. Nonetheless, it may be vital for these stakeholders to have immediate, reliable access to accurate information concerning ARRD activities for a particular clinical test. The present invention provides methods and systems for providing each stakeholder in a clinical trial relevant information and means to manage ARRD activities.

### System Configuration

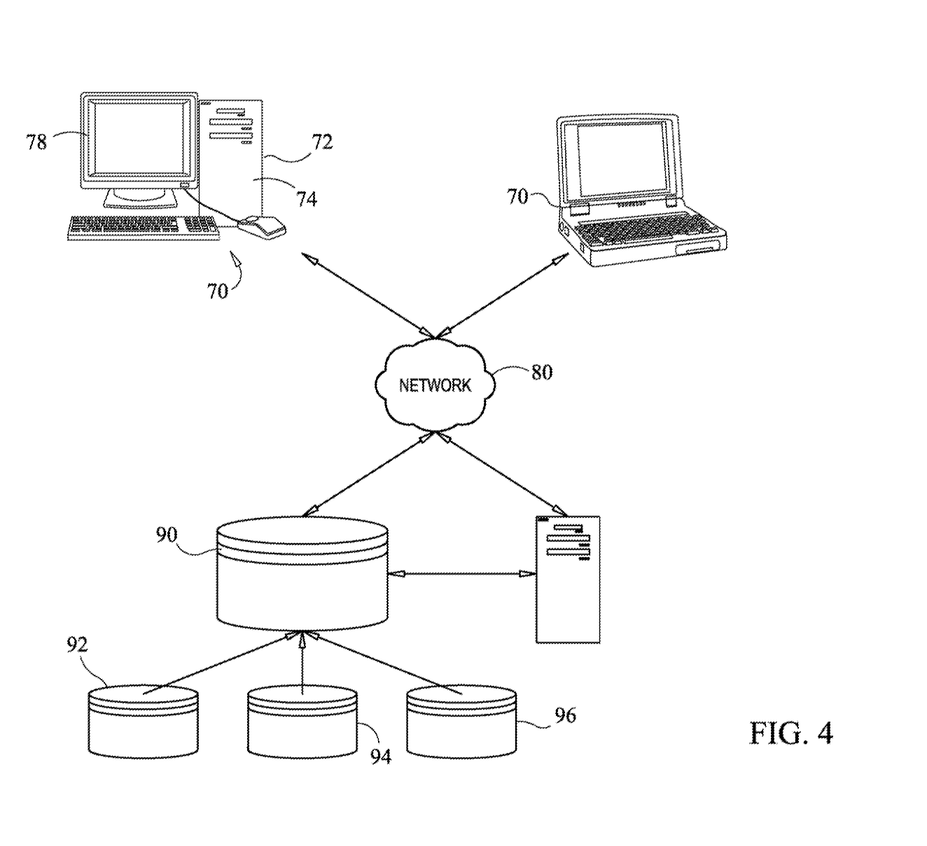

The systems and methods of the present invention may be used to provide stakeholders with the ability to manage ARRD activities. As seen in FIG. 4, an embodiment of the present invention employs typical hardware configuration architecture to provide stakeholders or users with information concerning ARRD activities. The computer system architecture further allows users with various functional abilities, including for example, viewing data, compiling data, data entry/modification, alerting/notification/commenting functions, and other functionalities.

With reference to FIG. 4, an embodiment of the computer system architecture is shown. As seen in FIG. 4, a user terminal 70 is provided. The user terminal 70 may be a general purpose computer or mobile computing platform. The user terminal contains a user processor or CPU 72, memory 74, and software or computer readable instructions 76. User terminal 70 may also contain a graphical user interface 78, such as a browser.

As seen in FIG. 4, user terminal 70 is connected to a network 80. Network 80 may be a local area network (LAN) or wide area network (WAN). Network 80 may be a private method, such as a virtual private network, or a public network, such as the Internet. As one of skill in the art would understand, user terminal 70 may connect to network 80 using a variety of methods and protocols. For example, user terminal 70 may use TC/IP protocol for transmitting and receiving data and instructions over the network. Additionally, user terminal 70 may be connected to network by any number of means. In an embodiment of the present invention, user terminal 70 may be connected to network 80 via a direct connection, such as a T1 line or fiber optic line. In alternative embodiments, user terminal 70 may be connected to network 80 using radio waves or other wireless means. In an embodiment, the system may provide more than one user terminal connected to a network using one or more protocols and/or connection methods.

With continuing reference to FIG. 4, the system architecture contains a database 90. Database 90 is generally a computer having a memory for storing data. Database 90 may also contain a processor for receiving and transmitting data. Database 90 may also use a processor for accessing data stored in the database memory. In alternative embodiments, as seen in FIG. 4, a server 100 (or other computer system connected to network 80) receives, processes, and/or executes instructions. Thus, server 100 may perform various logical functions and transmit data to and from user terminals 70 and database 80. In alternative embodiments, the system may contain more than one database or may contain ancillary databases 92, 94, and 96. Ancillary databases may store ancillary information to ARRD activities that may relevant to ARRD activities. In an embodiment, database 90 may store data related to ARRD activities, while ancillary databases 92, 94, 96 may store information related to the clinical trial study protocol, accounting, scientific information concerning the candidate drug, etc.

For example, in one embodiment, the system may be implemented using a well-known business and computer logic arrangements. In one embodiment, the user interface resides on a user computer and is a browser. A web application may reside on a web server with the web server connected to the user terminal via a network. The web application may be responsible for generating and processing information to/from the user terminal and user interface. The web server may also be connected via a network to an application server. The application server hosts an application that is used to negotiate connections, transmissions, or other processes of data and requests from the web server to the back end database.

The back end database may be a database server running a related database engine, such as Oracle® relational database system, a commercially available DB2 database, a Lotus® Domino TM server computer database, a Sybase® database, Microsoft® Structured Query Language (SQL) servers, and various Open DataBase Compliant (ODBC) databases.

In other embodiments, the system is implemented using a standalone software product that resides on the user terminal. The software may be executed from the user terminal and have synchronization functionalities that allow for the transmission and receipt of data and information over a network, which in turn may be connected to one or more servers and/or databases.

As one of skill in the art would understand, the specific configuration of the computer architecture is not vital to the system and methods described herein. Any number of different network configurations, computer platforms, and protocols may be used and nothing herein should be contrued as limiting the configurations employed in practicing the system and methods described herein.

### Software Overview

The system and methods of the present invention provide users and stakeholders in clinical trials with the ability to manage ARRD activities. The system and methods also provide users with different functionalities related to ARRD activities. The various users and stakeholders to which the different functionalities of the systems and methods presented herein may be offered include, but are not limited to Sponsors, Trial Managers, Operations Personnel, CRA/Monitor, Site Personnel, and Depot Personnel.

As one of skill in the art would understand, the type of stakeholders or users of the system may vary and certain duties and responsibilities of managing a clinical trial may vary from example to example. Accordingly, in the following description it should be understood that any functionality or responsibility described in connection with a particular user may, under different circumstances, fall with a different user or stakeholder.

One stakeholder or user of the system is the Sponsor and/or Trial Manager. Sponsors typically assume the financial and legal responsibility for conducting a clinical trial. Sponsors often have vested interests in the clinical trial and usually own rights to manufacture or distribute the candidate drug being studied. Sponsors often use a Trial Manager to design and oversee a clinical trial. Trial Managers may be an employee of the Sponsor or, alternatively, the Sponsor may contract for a Trial Manager's services. Sponsors and Trial Managers usually have input and responsibility for the design of the clinical trials and provide a protocol that designates the various criteria used during the study in question. These criteria may include, but are not limited to subject qualification, number of subjects needed, type of subjects needed, timeline of the study, dosage levels, treatment regimen, etc.

With respect to ARRD activities, Sponsors and Trial Managers usually retain overall responsibility for said processes but do not directly oversee them as those tasks often fall on other individuals. Nonetheless, Sponsors and Trial Managers maintain a high interest in ARRD activities as they may affect the success of regulatory approval and/or regulatory compliance. More particularly, a Sponsor's primary interest with respect to ARRD activities concerns maintaining patient safety with respect to medication dispensation and use, minimizing audit findings, streamlining data collection and reporting, and ensuring the integrity of the clinical trial process. Trial Manager's interests are aligned with the Sponsor. Thus, summary level output data reporting functionalities, complete chain of custody reporting for every pack throughout its lifecycle, and other appropriate summary statistics and information are of interest to Sponsors and Trial Managers.

Accordingly, in an embodiment of the present invention, reporting parameters and requirements are associated with Sponsors and Trial Managers such that they may receive, retrieve, view, compile, or otherwise obtain accurate clinical trial data relating to ARRD activities. In an embodiment of the present invention, the data may be provided in a variety of formats, including text, graphical displays, XML, portable document formats, etc. The reporting may be provided via any number of conventional means including paper reporting, electronic messages, bulletin style message boards, etc. In an embodiment of the present invention, Sponsors and Trial Mangers may log on to the system and perform query activities to obtain data as described in more detail below.

In another embodiment, complete chain of custody information concerning every treatment unit or medication unit used in a clinical trial can be reported in summary and detail form for submission with the final clinical findings. In another embodiment, Sponsors and Trial Managers may receive intermittent reporting data concerning ARRD activities. In this embodiment, users may utilize the reports to change the design of the current clinical trial protocol, or, alternatively, use the reports to implement changes in future clinical trial designs. In another embodiment of the present invention, the ARRD activities performed by the methods and systems disclosed herein may be integrated into existing CTMS systems to complement or otherwise complete reporting and analysis functions.

Another stakeholder or user of the system is the clinical research associate or Monitor. Monitors are individuals either employed by a CRO or Sponsor to help administer and oversee various processes of a clinical trial. For example, a Monitor may be responsible for more than one clinical trial and may visit a number of investigative sites during the performance of her duties. Monitors typically report to a clinical director within the CRO as well as the Trial Manager for each study they are monitoring. Monitors often spend time in the field visiting investigative sites. A Monitor's duty may include various activities including (a) site initiation (training of site staff on protocol and data keeping), (b) site monitoring (checking common inconsistencies or data errors and verification of data entered against source), and (c) drug reconciliation and returns (confirming return of medication and packaging medication for site).

In an embodiment of the present invention, the system provides a Monitor with the ability to view, record, and validate ARRD data on site. In this embodiment of the invention, the system provides the Monitor with the ability to view relevant data attendant to a clinical trial onsite at an investigational site. In this manner, the Monitor has access to all relevant data for a clinical trial in a convenient and organized fashion. In alternative embodiments, the system provides the Monitor with the ability to request, extract, or otherwise parse certain data parameters based on a query. In alternative embodiments, the Monitor may enter data into the system while at the investigative site. The data may then be recorded in the system database in real time. In this embodiment, the Monitor may use a mobile computing platform, such as a laptop or personal digital assistant, connected to a network that is capable of transmitting and retrieving ARRD data.

In alternative embodiments, the system may provide the Monitor with the ability to enter, view, modify, or otherwise request ARRD data with mobile computing platform even when not connected to the network. In this embodiment, the mobile computing platform may have a memory in which ARRD data for one or more clinical trials or clinical sites is stored. The Monitor may then view, edit, and record information while at the investigative site with the ARRD data being displayed and stored on the memory of the mobile computing platform. At a later point in time, the Monitor may then connect the mobile computing platform, or in alternative embodiments a detachable memory unit, to a network. The system may then synchronize new and revised data with existing data in the system database.

In one embodiment, the system will include synchronization rules and procedures designed to ensure the integrity and source of the data. For example, in one embodiment only data for which the Monitor has permissions to edit will be written into the database during synchronization. Each piece of new or revised data may require verification by the Monitor during the synchronization process to ensure its reliability. Changes made to the database since the download of a local version will be indicated for acceptance/rejection during synchronization. This offline working may be achieved via a local version of the ARRD software, or a download into a viewer or other application such as a spreadsheet.

In an alternative embodiment, the system may provide the Monitor with error checking or validating functions. For example, in one embodiment upon entering a medication unit count or editing of other ARRD data, the system may ask for confirmation by the Monitor before storing the new entry in the database, or may ask for a query to be raised for the Investigator to reconfirm their entries. In some instances, this validating feature may occur immediately after the Monitor has entered data, such as in the case that the Monitor is using a computing system that is connected to a network. In alternative embodiments, the error-checking feature may prompt confirmation of data change prior to storing the change in the system. This may occur at later times, such as the case where the new data is temporarily stored on a mobile computing platform before synchronization within the database of the system.

In an embodiment of the present invention, the Monitor may also compile, request, or otherwise execute reporting functions. The reporting functions may be of any type suitable for fulfilling a Monitor's reporting duties. Thus, in an embodiment of the present invention, a Monitor may be required to report all discrepancies of a medication unit count for a particular clinical trial at a particular investigative site. In this example, the Monitor may reconcile returned treatment packs and medication units at an investigative site. For each discrepancy, the Monitor will verify the true count, enter the revised data into the system, and raise an electronic query for the study site personnel. Study site personnel will reply to open queries, and this may result in subsequent changes to the data captured—either in the study site personnel's records or Monitor's records. All previous versions of the data and their changes will be captured within the audit history. Upon completion, the Monitor may query the system for a report of all modified or edited pack or medication unit count changes and be provided by the system a report containing data related to said discrepancies. The report may then be sent to appropriate personnel or stored within the system for later retrieval or viewing.

In another embodiment, the system may provide a Monitor with a “to do” list. For example, the system may provide the Monitor or other stakeholder with information particular to the stakeholder's responsibilities attendant to the study. Thus, a Monitor may be provided with various reports or lists that show sites to visit based upon priority, sites at which treatment packs are due to be returned but have not yet been accounted for by study site personnel, treatment packs accounted for but not yet reconciled, packs with outstanding reconciliation discrepancies/queries, or a list of treatment packs that are ready for destruction. As one of skill in the art would understand, the commenting/reporting features may be automated or may be initiated by another user of the system.

In an alternative embodiment of the present invention, the system may provide templates or other populatable forms. In this embodiment of the present invention, the Monitor—depending on the specific clinical study protocol or other considerations—may be required to provide documentation for certain activities. Thus, in one example, the Monitor may be required to provide documentation during return processing displaying the unique identifiers for each treatment pack in a consignment and instructions concerning the proper disposal of the treatment packs. Where the study protocol has defined certain parameters for inclusion in consignment returns processing, a form or other populatable template may be provided for the system. Upon instructing the system, the Monitor may be provided said documentation with accurate and timely data.

As one of skill in the art would understand, the system may be highly configurable to allow unique access, reporting, and data entry variability. In this sense, a particular Monitor may have access to certain data entry and reporting functions for a particular clinical trial. Moreover, the same Monitor may have different data entry and reporting functions for different clinical trial and/or investigative sites. In this sense, the study protocol or other considerations may dictate the capabilities and system configurations along a number of parameters. These considerations may vary by user, investigative site, clinical trial protocol, date, and/or other factors and/or by some combination of the aforementioned factors.

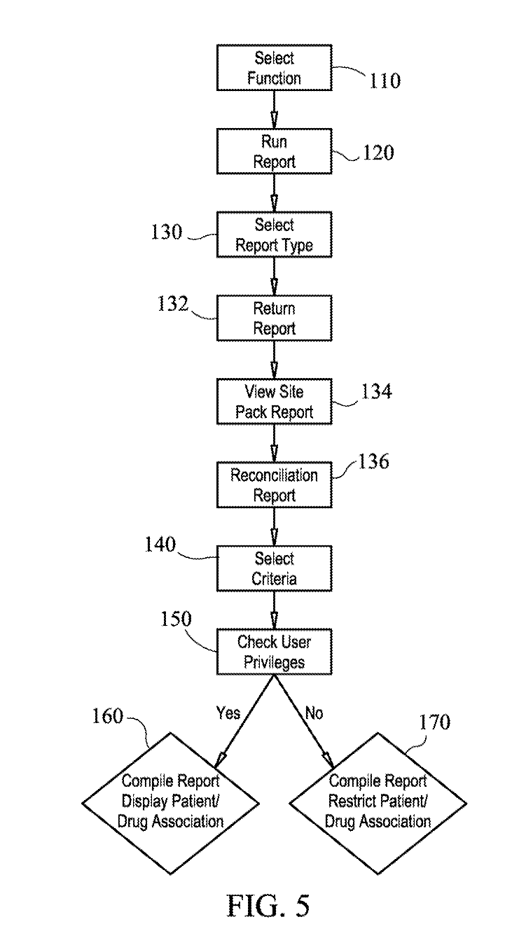

In one embodiment, the reporting function may limit the ability of a Monitor to create reports identifying which subjects received specific treatment drugs. In this embodiment, the study protocol may be a “triple-blind” study in which neither the Sponsor, Trial Manager, or Physician (or other site personnel) is permitted per study protocol to know whether a subject is taking or has received drug or placebo. As seen in FIG. 5, the system may provide a Monitor with the ability to select a function 110 with one of said functions being the ability to run a report 120. Upon selection, the system may prompt the user to select from a variety of reports 130, such as a return report 132 site pack report 134, or reconciliation report 136. Upon selection of a report, such as a reconciliation report 136, the user may be prompted to select from a variety of relevant criteria in a select criteria step 140. Such criteria may include, for example, date ranges, site selection, clinical study identification, etc. Additionally, the system may check to determine whether a user should be restricted from seeing certain data. While this may occur at any point in the process, in this example, the system may check to see whether the user is authorized to view patient information in association with drug received. In blinded trials, certain users, such as a study site personnel or trial managers, should not see unique patient identifiers in association with drug received. Thus, after selecting criteria in step 140, the system may check a user's identification against a rule set, such as a privilege rule set 150. Typically, user privileges may be set by operator personnel during the design of the system. If the user privileges allow for the user to see patient data in association with drug received, then the system may provide the user with a report displaying patient/drug association 160. If the user is not permitted to see such an association, the system may restrict out identifying information, such as patient name or patient ID 170. As one of skill in the art would understand, any number of different functions and criteria may be used to generate customizable reports with and without data fields. In this sense, the present invention is capable of providing all stakeholders with accurate and relevant data depending on the specific needs of the stakeholder, the protocol of the study, or other relevant considerations.

In an alternative embodiment, such selection criteria may expire or otherwise no longer be applicable. In this sense, the system may be configured to change restrictions of certain functions depending on the happening or non-happening of certain events. Using the above example, once clinical trials have been completed, the need for a triple blind study may no longer be applicable. Thus, the system may be configured (either from inception or modified at a later time) to create reports displaying patient/drug associations after the completion of the clinical trial. As one of skill in the art would understand, a variety of different combinations, triggering events, restrictions, and stakeholder filters may be employed depending the specific requirements of a stakeholder, clinical study protocol, or other factors.

Site personnel may be additional users or stakeholders of the system. Site personnel may include various individuals including one or more investigators, one or more site nurse practitioners, one or more study site coordinators. With respect to ARRD activities, site personnel are typically involved in the accountability processes. Namely, site personnel are responsible for receiving and logging treatment packs, recording dispensation, and recording the return of medication units and treatment packs including recording the quantity of returned medication units within each treatment pack. Accordingly, site personnel must be able to accurately record, view, and otherwise manage data related to treatment packs and medication units for a clinical trial.

In an embodiment of the present invention, the system provides site users with the ability to log, record, store, access, view, modify, or otherwise manage data related to ARRD activities. More particularly, site users may use a used terminal to enter data concerning accountability processes and coordinate their work by reporting activities that require attention, such as packs due back from patients or outstanding reconciliation discrepancies for resolution. The entered data may then be recorded in the system database. A variety of different functions may be provided to site personnel including the ability to view reports, run queries, edit data entries, etc. As discussed in more detail below, a variety of different capabilities may be provided to the different stakeholders. Moreover, each clinical trial, site, CRO, or other entity may have differing access to functionalities and information as determined by the operations user, clinical study protocol, or other factors.

In one embodiment, site personnel may be able to associate text entries with a data field, such as a particular treatment pack or test subject. In this embodiment, site personnel may attach a comment that is associated with another data field in the system. In another embodiment, a user may attach a query that is associated with a data field in the system. As one of skill in the art would understand, the comment and/or query could be made for any number of reasons, including a reminder to the user themselves or as a message to a different user of the system. Thus, in one embodiment, a user may post or enter a comment that will be displayed to one or more users of the system. In another embodiment, a comment and/or query may be displayed to a select group of users. In another embodiment the system may prompt the user to enter a comment explaining the reason for the change when a piece of data previously entered and saved has been changed. In one embodiment, a first user may enter a comment associated with a particular subject, treatment pack, or other data field. A second user may then see the comment, prompting action, whether it is a reconciliation process, return process, or simply a response to the first user's comment. As one of skill in the art would understand, the ability to configure the system to selectively allow comments and/or queries to be entered and/or displayed to one or more users of the system provides a robust and information rich system for managing ARRD activities in clinical trials. As used herein, comment may be used generically to refer to a text entry or other notation associated with data stored by the system. Thus a comment may be an order, a request, a query, a notation, or any other form of entry within the system by a user that is attached or associated with data stored in the system.

Depot personnel may also be stakeholders and users of the system and methods present herein. Depot personnel include any individuals that manage, oversee, or are involved in the returns process (and in some cases the destruction process) during a clinical trial. Depot personnel are typically responsible for ensuring the proper receipt of return consignments and their storage and eventual disposal. Accordingly, users of the systems and methods presented herein are provided data management functionalities to ensure the proper logging, routing, and recording of candidate drug shipments.

Operations personnel may also be stakeholders or users of the system. While typically not directly involved in ARRD activities or clinical trial management, operations personnel are responsible for designing, selecting, authorizing, and otherwise configuring the various system parameters according to each particular situation. Thus, operations personnel may work in conjunction with a Trial Manager and the clinical study protocol to design the appropriate data fields, site identifiers, data constraints, and other parameters that are to be employed in any particular system. In some instances, the operations personnel may be an employee of a Sponsor or a CRO. In other instances, operations personnel may be a consultant or other individual. In some instances, operations personnel may also serve as service technicians in helping the various other stakeholders or users of the system with the methods and systems disclosed herein. Operations personnel may also be referred to as administrators.

In an embodiment of the system, user privileges may be assigned to different stakeholders of the system. Thus, in an embodiment of the present invention the operations personnel may assign individual users with different privileges. In an embodiment of the present invention, privileges may provide data viewing privileges, report privileges, data modification privileges, commenting or querying privileges, etc. Thus, in this manner, each user of the system may be provided individual level permission to various functionalities and data management activities in the system. In an alternate embodiment, user privileges may be assigned to stakeholder groups or types. Thus, for example, all site personnel may be provided with the same privileges to view and enter data, but they may be restricted from editing entered data. In alternative embodiments, an Investigator may be provided the additional privilege of creating reports. As one of skill in the art would understand, privileges may be assigned to different users of the system to ensure accurate data entry, secure the integrity of the system data, as well as comport with the clinical study protocol, i.e. blinded studies, etc.

### System Capabilities

In an embodiment, the system and methods of the present invention provide full ARRD activity management. As discussed previously, ARRD activities include accountability, reconciliation, returns, and destruction.

In an embodiment of the present invention, the system may be designed to fulfill activities related to ARRD processes. Thus, during the design of the system, ARRD activities may be identified. Additionally, each ARRD activity may be designed with requirements. These requirements identify the needed capabilities of the system. Thus, a Trial Manager or Sponsor in conjunction with operations personnel could define parameters within the system to meet the various design requirements for a particular clinical trial or stakeholder.

In the following example, the system is designed with various requirements. Attendant to each requirement, the system may provide one or more users functionality for meeting the requirement of the system. Additionally, in the following exemplary embodiment, the design stakeholders may identify or assign one or more stakeholders to a particular requirement/functionality. As described previously, rule sets of the system may provide different users with different capabilities.

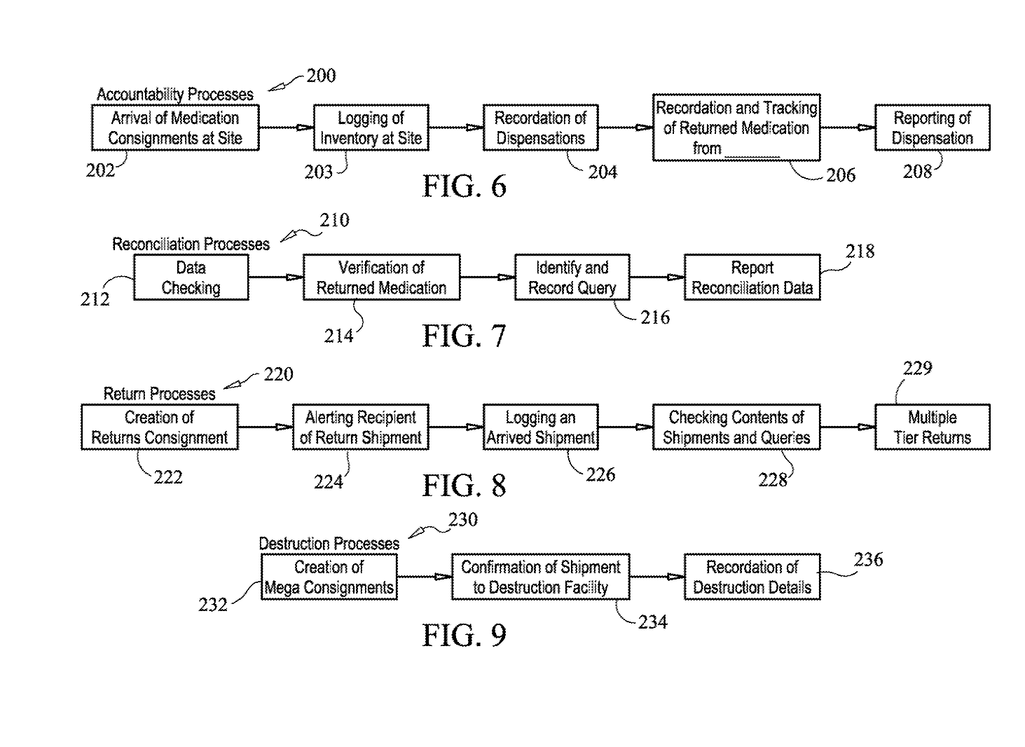

As seen in FIG. 6, in one embodiment the system and method provides management of data related to accountability processes 200, including (a) arrival of medication consignments at site 202, (b) recording or logging of inventory at the site 203, (c) recordation of dispensation 204, (c) recordation and tracking of returned medication including number of medication units returned from subjects 206, and (d) reporting of inventories and/or dispensation 208.

With respect to the arrival of medication consignments at the site, system requirements may include:
linking system with existing drug supply management/ordering systems or clinical trial management systems or supply chain management systems including interactive voice response (IVR) systems and interactive web response (IWR) systems.
displaying site inventory details including pack status and associated data such as consignment number, pack number, arrival date, batch number, expiry date, etc.

With respect to the reporting of medication arrivals, the system may be designed to provide a number of different capabilities including:
Provide paper/electronic reports detailing the current inventory and inventory history for site use.

With respect to the recordation of dispensations, system requirements may include:
Linking the system with randomisation and supply chain management system databases including IVR and IWR systems to receive pack allocation information.
Display site dispensing log
Record actual dispensing dates, times, patient details and site personnel performing the dispensation.
Confirm identity of unit dispensed
Record quantity dispensed
Electronic signature

With respect to the recordation and tracking of returned medication from subjects, system requirements may include:
Record and log the receipt of returned packs
Record the quantity of medication remaining in the returned packs
Calculate and display the expected quantity of medication units returned with reference to the dispensation date, treatment pack return date or subsequent repeat dispensation date, and the dosing regimen as determined by the study protocol.
Perform data checks on validity of information entered.
Provide prompts to the user when discrepancies arise
Record comments from the user against data points.
Provide reporting that displays outstanding packs
Electronic signatures

With respect to the reporting of dispensation, the system may be designed to provide a number of different capabilities including:
Provide paper/electronic reports detailing the dispensing log for site use.

With reference to FIG. 7, in an embodiment the system and method provides management of data related to reconciliation processes 210, including (a) data checking 212, (b) verification of returned medications 214, (c) identify and record query 216, and (d) report reconciliation data 218.

With respect to data checking, the system and methods of the present invention are designed to eliminate or reduce discrepancies by at least (a) identifying packs not returned, accounted for, or not reconciled for either by the site personnel or monitors or other stakeholders or (b) automatically identifying, flagging, and notifying stakeholders concerning discrepancies of treatment pack count by stakeholders. In an embodiment of the present invention, the system provides for data checking by ensuring that dispensation data and medication receipt data are either linked or otherwise reconciled during data entry by site personnel, monitors, or other stakeholders. As one of skill in the art would understand, a variety of data checking methods may be implemented to ensure that site and Sponsor documentation is maintained in accordance within regulatory guidelines at all times.

With respect to verification of returned medication, system requirements may include:
Log monitor reconciliation activity
Allow Sponsor to determine a fixed percentage to be checked
Allow system to select units to check in relation to defined percentage
Enable local offline monitor use whilst at study site
Automatic flagging of treatment packs where a discrepancy between site and Monitor tablet counts occurs—prompting the raising of a query by the Monitor,
Electronic signature

With respect to the identification of and resolution of queries, the system requirements may include:
Allow the monitor to raise a query against any particular returned treatment pack.
Allow the investigator or other site designated personnel to respond to a query
Allow queries to be identified as closed either once they have been answered by the site, or when physically marked as such by the monitor,
Monitors must be able to raise a subsequent query around a pack if the answer to the previous query was inconclusive.
Full audit trail of changes to data with reasons for change should be visible when desired
List current queries and their status
Electronic signature

With respect to reporting, the system requirements may include:
Identification of units returned to site
Identification of units not logged as returned that are not in use by the subject
Identification of units returned and reconciled
Identification of units associated with outstanding queries
packs due for return but not accounted for by the Investigator
packs accounted for but awaiting reconciliation
packs with outstanding reconciliation discrepancies/queries
packs ready for return to depot
summary level progress data such as proportion of packs reconciled, returned, destroyed etc.

As seen in FIG. 8, in one embodiment the system and method provides management of data related to return processes 220, including (a) creation of a returns consignment 222, (b) alerting a recipient of a return shipment 224, (c) logging an arrived shipment 226, and (d) checking contents of shipment and queries if necessary 228, and (c) allowing for multiple tier returns 229.

With respect to the creation of a returns consignment, system requirements may include:
Enable selection of returns consignment destination
Create a consignment from the set of reconciled and unused/expired/damaged packs at site. Automatically create a consignment number or enable the user to specify a consignment number.
Create consignment documentation to accompany physical shipment
Delete or edit a consignment
Electronic signature
Enable local offline monitor use whilst at study site.

With respect to alerting a recipient of a return shipment, system requirements may include:
Provide automated alert of consignment for example via fax, email, text message, system prompt or automated outbound call

With respect to logging an arrived shipment, system requirements may include:
List outstanding consignments for location
Record arrival date/time
Alert monitor and other stakeholders that shipment has arrived for example via fax, email, text message, system prompt or automated outbound call.

With respect to checking contents of shipment and queries, system requirements may include:
Provide the affiliate/depot with a consolidated report containing the status of each unit that has been identified for shipment and associated information
Allow the depot/affiliate to verify and log and record the contents of the shipment including (if required) a report count of medication units within each treatment pack
Allow the depot/affiliate to raise a query against any particular treatment pack entered by the monitor and allocate the query to a user or users.
Allow the monitor or other designated personnel to respond to a query (allow free text and possible list to select from)
Allow queries to be identified as closed either once they have been answered by the site, or when physically marked as such by the monitor or depot/affiliate.
Full audit trail of changes to data with reasons for change should be visible when desired
Depots must be able to raise a subsequent query around a pack if the answer to the previous query was inconclusive
List current queries and their status assigned to a monitor
Electronic signature

With respect to allowing multiple tier returns, system requirements may include:
Accommodate single and two-tier returns supply chains such that medication may be returned to a single depot before destruction/shipment for destruction, or returned first to a local depot then to a second depot prior to destruction/shipment for destruction.

As seen in FIG. 9, in one embodiment the system and method provides management of data related to destruction processes 230, including (a) creation of mega-consignments 232, (b) confirmation of shipment to destruction facility 234, and (c) recordation of destruction details 236.

With respect to the creation of mega-consignments, system requirements may include:
Enable selection of returns mega-consignment designation
Create a mega-consignment from the set of consignments received from individual sites or depots and any unused/expired/damaged packs at depot. Automatically create a (mega)consignment number or enable the user to specify a (mega)consignment number.
Create (mega)consignment documentation to accompany physical shipment
Delete or edit a mega-consignment.
Electronic signature

With respect to confirmation of shipment to destruction facility, system requirements may include:
Record shipment date and time

With respect to recordation of destruction details, shipment requirements may include:
Record the destruction certificate details against mega-consignment including certificate number, time and date.
Record the destruction certificate details against any consignments and individual treatment packs contained within the mega-consignment including certificate number, time and date
Record the destruction certificate details against any treatment packs contained within each consignment within the mega-consignment including certificate number, time and date
Electronic signature
Add a comment against the destruction certificate information
Issue notification that destruction has occurred to interested parties

In general, a number of overall system requirements may be implemented. Accordingly, a variety of functionalities may be provided to a stakeholder of the system, including:
integration of ARRD functionalities in non-IVR/IWR supported studies whereby pack-list, randomisation, patient enrolment and medication supply shipment information will be entered/imported into the ARRD solution rather than directly accessed from an IVR/IWR database/data file transfer.
ability to confirm data entry, point by point and/or session by session
secure system including password/login and secure data transmission
print screen and other printing capabilities, in particular for hard copies of site inventory and dispensing logs
automated data check of data for sense, logic, and appropriateness
data stored and provided in usable, reportable formats
configurability to specific study requirements
use of system platform across studies and/or Sponsors, for example to combine return shipments from multiple studies into a single return consignment, and apply a destruction certificate relating to consignments for multiple studies to packs within each corresponding study.
Manage the ARRD activities for situations where more than one study is operating using a common (shared) pool of supplies
deleting or editing a return consignment
removing certain packs from an individual consignment to be accounted for individually (undo consignment).
manage open label, pack numbered and subject numbered supplies
manage supplies uniquely labelled with barcodes or RFID tags
create consignments and mega-consignments uniquely labelled with barcodes or RFID tags
address instances of unit destruction when units allocated to a different study
allow “immediate” destruction of units after use
allow destruction of units and packs by site
allow for packs lost or damaged after dispensation to the patient
allow use of system even if subset of sites are not using system, for example when certain sites maintain paper dispensing logs requiring monitors privileges to enable them to copy site data into the system in addition to performing and recording reconciliation activities for those specific sites only.
export certain ARRD data in scheduled or real-time to CTMS and DSMS and other eClinical software solutions
allow Sponsors, CROs, depots and other stakeholders to technology transfer the solution to operate independently upon clinical trials they are responsible for.

As one of skill in the art would understand, the various requirements and functionalities listed above are exemplary only, and each particular use of the system and methods provided herein may contain all, none, or some of the various requirements detailed above. The system and methods provided herein may be used to manage ARRD activities and may be specifically tailored to meet the different requirements of a particular stakeholder or clinical trial. In an embodiment of the present invention, a Sponsor or trial manager may work with an administrator or other group of individuals to define parameters of the system based on the requirements of a clinical trial.

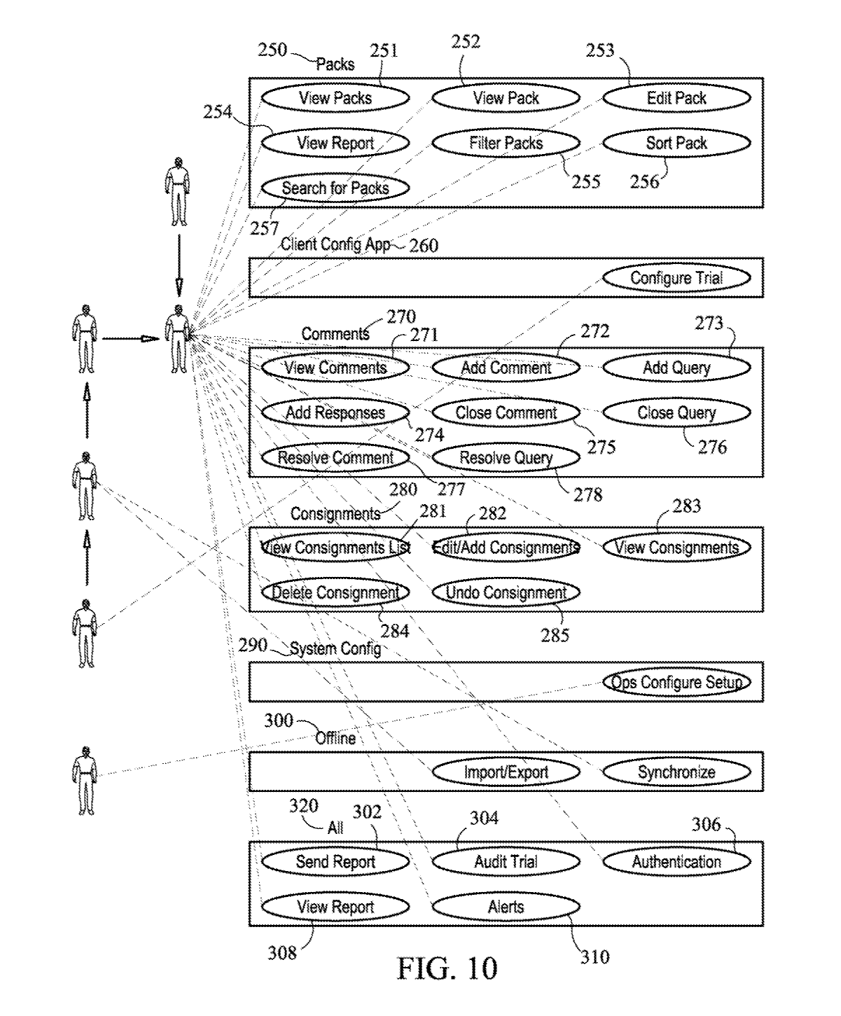

With reference to FIG. 10, a system is provided wherein a number of the above described requirements are implemented in computer based architecture. As seen in FIG. 10, different stakeholders of the system may be provided with access to different functionalities of the system. With continuing reference to FIG. 10, stakeholders may include Operations Personnel, Trial Manager/Sponsor, CRA/Monitor, and Site Personal.

As seen in FIG. 10, the system provides each stakeholder with different functions. In some instances, the system may provide more than one stakeholder type with access to the same functions. In other instances, only one stakeholder may be provided access to a function. As seen in FIG. 10, functions of the system may be grouped into different types. Thus, as seen in FIG. 10, an embodiment of the system may provide users with functions relating to packs 250, client configuration 260, comments 270, consignments 280, system configuration 290, and offline functionalities 300.

Within each group type, an embodiment of the system may have a variety of different functionalities. For example, with respect to pack processes 250, a user may be able to view pack 251, view packs 252, edit pack 253, sort packs 254, search for packs 255, view report 256, and filter packs 257. These functionality will also be available by patient number, site, consignment number etc. As one of skill in the art would understand, the different functionalities and data capabilities of each process or functionality may be designed to manage ARRD activities.

In another embodiment of the methods and systems presented herein, the trial configuration functionalities 260 may allow a system administrator to define the parameters and requirements of the system. Thus, the system may be able to define parameters, such as pack data or consignment data in such a fashion to specifically take into account the particulars of a clinical trial. Thus, the data fields and data types of a particular system is tailored to the clinical trial parameters, including such things as medication unit type, dosage units, blindedness of the study, etc.

As seen in FIG. 10, comment processes 270 may include view comments or notes 271, add comments 272, add query 273, and add response 274, close comment 275, close query 276, resolve comment 277 and resolve query 278. Thus, a user of the system may be able to post a comment for other users to see. The comment may be associated with a pack or consignment or other data type. A second user may then view the comment and take action, if necessary. Such action may include checking the validity of the data entered, recounting medication units, or other action. The second user may then edit the data entered or instruct the first user or another user to edit the data, make no data change, or other conduct some other action.

With respect to consignment processes 280, in an embodiment of the present invention a user may be able to view consignment list 281, edit or add consignment lists 282, and view consignment 283, delete consignment 284, undo consignment 285. In this manner, one or more users may have access to a variety of data regarding consignments and may track, edit, or otherwise take actions regarding consignment activities in a clinical trial.

As further seen in FIG. 10, an administrator may be given system configuration processes 290 to define the various parameters of a system. In this fashion, an administrator may define rule sets or other logical instructions in accordance with the particulars of a clinical trial. The administrator or other user may also assign different users with permission level access to different functionalities of the system, including such things as commenting privileges, query privileges, reporting privileges, data modification privileges, etc. Additionally, an administrator may define other rule based events such as scheduling reports based on timing intervals or other triggers. Also, and administrator may configure the system for integration with other IVR. CTMS or DSMS systems as described in more detail below.

With respect to offline processes 300, the system may provide one or more users with the ability to record, view, and edit ARRD activity data on a computing platform, such as a mobile computing platform, while not connected to a network. In this fashion, the system may import/export functionality to synchronize data entered into a mobile computing platform while the mobile computer is not connected to the network. This functionality enables users of the system to record, view, and edit ARRD activity data while not connected to the network. As one of skill in the art would understand, a user may download data for a study, various site data within one study, or site data relating to more than one study prior to disconnecting from the network. The user may then participate in ARRD activities while offline, including for example, visiting sites. Upon connecting to the network at a later date, the user may synchronize newly acquired or modified data with data stored on the database. In one embodiment, the system may automatically update any changes made offline into the main or central database. In other embodiments, the system may require manual confirmation by a user before updating the main database. In other embodiments, only some data types may be changed depending on the privileges of a user, whether done offline or online. In alternative embodiments, certain data type might be automatically updated or synchronized, while other data types may require stakeholder intervention, such as confirmation of change or the approval of another stakeholder. Such variations may be implementing using rule sets that govern a user's ability to modify data, enter data, or otherwise access functionalities of the system. The rule sets may be particular to a user or a user class.

As seen in FIG. 10, other general functionalities may be provided by the system including, send reports 302, audit trail 304, authentication 306, view report 308, and alerts 310. Each functionalities or process is an additional feature that helps the users of the system manage ARRD activities. For example, the audit trail 304 functionality allows a user to view and report current and historic data values against specific packs, alongside information regarding who made the change, the time/date of the change and the reason for changing the data. In another example, alert functionality 310, may provide one or more users with a notification of the happening or non-happening of an event, such as a discrepancy in medication count or the failure of a depot to confirm shipping of a consignment to a destruction facility.

As seen in FIG. 10, the stakeholders of a clinical trial may be given varying access to the different functionalities of the system. Thus, for example, operations personnel 312 may only be given access to the system configuration functionality 290. In this manner, an administrator or other individual can control or assign access to the different functionalities of the system u sing rule sets or privileges.

As further seen in FIG. 10, the trial manager or Sponsor 314 may be provided access to the trial configuration functionalities to tailor the system to a particular clinical trial. The CRA/Monitor 316 may be provided access to offline usage of the system in order to conduct ARRD compliance activities on site. And the site users may be provided access to pack functionalities 250, comments functionalities 270, consignment functionalities 280, offline functionalities 290, and general functionalities 320. In this sense, a group of stakeholders may each be given access to different functionalities of the system. Thus one or more type of user may be able to add and edit data, while a different type of user may be able to view data only. In this manner, the system provides complete flexibility and tailoring of access to system functionalities for monitorying AARD activities of a clinical trial. As one of skill in the art would understand, any number of different users may be provided access to any number of different functionalities and system processes. Nothing contained herein should limit the present invention to the particulars of the examples described herein.

### Integration Capabilities

Another feature of the systems and methods presented herein is the ability to fully integrate the management of ARRD activities into existing eClinical software solutions such as trial supply management systems including Interactive Voice Response (IVR) and Interactive Web Reports (IWR) systems, clinical trial management systems (CTMSs), drug supply management systems (DSMSs) and electronic data (EDC) systems. As discussed previously, a number of management systems exist to manage clinical trials and supplies. In some instances, rather than replacing CTMS and DSMS systems, the present invention allows users to integrate ARRD management with existing CTMS and DSMS systems.

CTMS systems typically only provide access to Sponsor personnel. CTMS systems usually contain study design details, site and investigator details, tracking of payments, tracking regulatory and ethics approvals, treatments studied, key milestone dates, study progress tracking and reporting, and monitoring meeting planning and reporting. DSMS systems enable Sponsors to track and manage supplies of a particular study. Typically, the supplies are managed from manufacture to delivery to a particular study site and allows batch definition, packaging design, labeling, pack-list creation, expiry date management, site details and shipping addresses and ordering and shipment management. EDC systems usually enable Investigators to record clinical data collected during patient assessments electronically including efficacy and safety parameters as specified by the study protocol and may include entry of certain dispensing information associated with each patient visit.

In an embodiment of the present invention, the system and methods herein are able to provide CTMS systems with ARRD management processes. In another embodiment of the present invention, the system and methods herein use data from CTMS systems and integrate the data into the ARRD system. In another embodiment of the system, the CTMS system may both receive and send data to the ARRD system and the ARRD system may both receive and send data to the CTMS system. In this fashion, the data and activities tracked by each system are synchronized, with each system incorporating data from the other system. The above embodiments hold true for the possible data sharing between ARRD and DSMS and ARRD and EDC systems. In alternative embodiments, the system is capable of displaying ARRD activity data to a user from multiple format in a single view. Thus, the system may display to a user data from a CTMS system, IVR system, paper based system, etc. all within a single view, whether the systems are being used in one study or multiple studies.

In other uses, the systems and methods herein may be integrated into existing interactive voice response systems (IVRs). IVR systems are often used to manage aspects of clinical trial supply chain, up to the point of allocation of treatment packs to a specific patient. Thus, as in the case with CTMS and DSMS systems, the system and methods of the present invention may be integrated into existing IVR solutions. In this fashion, data from an IVR database, such as pack list information, depot and site inventories, re-stocking shipment information, patient or subject medication pack allocations, data may all be sent to and from the IVR to the ARRD system described herein. In other embodiments, the system and methods provided herein may exist as a stand-alone product to manage ARRD activities. Such products may include software residing on user computers or through a web based solution to manage ARRD processes.

In other uses, the system described herein may be used to manage ARRD activities for more than one clinical trial. In this embodiment, even where each clinical trial is being conducted with different management systems, the system herein supports integration of data from various sources and provides solutions for managing data in one convenient format. Thus, for example, a Sponsor may have more than one clinical trial being managed by a contract research organization. Additionally, the same Sponsor may have another clinical trial managed using an IVR protocol and another not using IVR trail supply management. In this example, the system is capable of integrating data from the different sources e.g. CRO, IVR into one system for viewing, tracking, reporting, editing, etc and is capable of combining data across studies where sites and depots are common to more than one study—for example, creating a mega-consignment for destruction consisting of consignments from more than one study. In this manner, trial directors and depot personnel, for example, are provided a single solution for managing ARRD activities across a variety of platforms that may be involved in conducting and managing different clinical trials.

### EXAMPLES

### Example 1

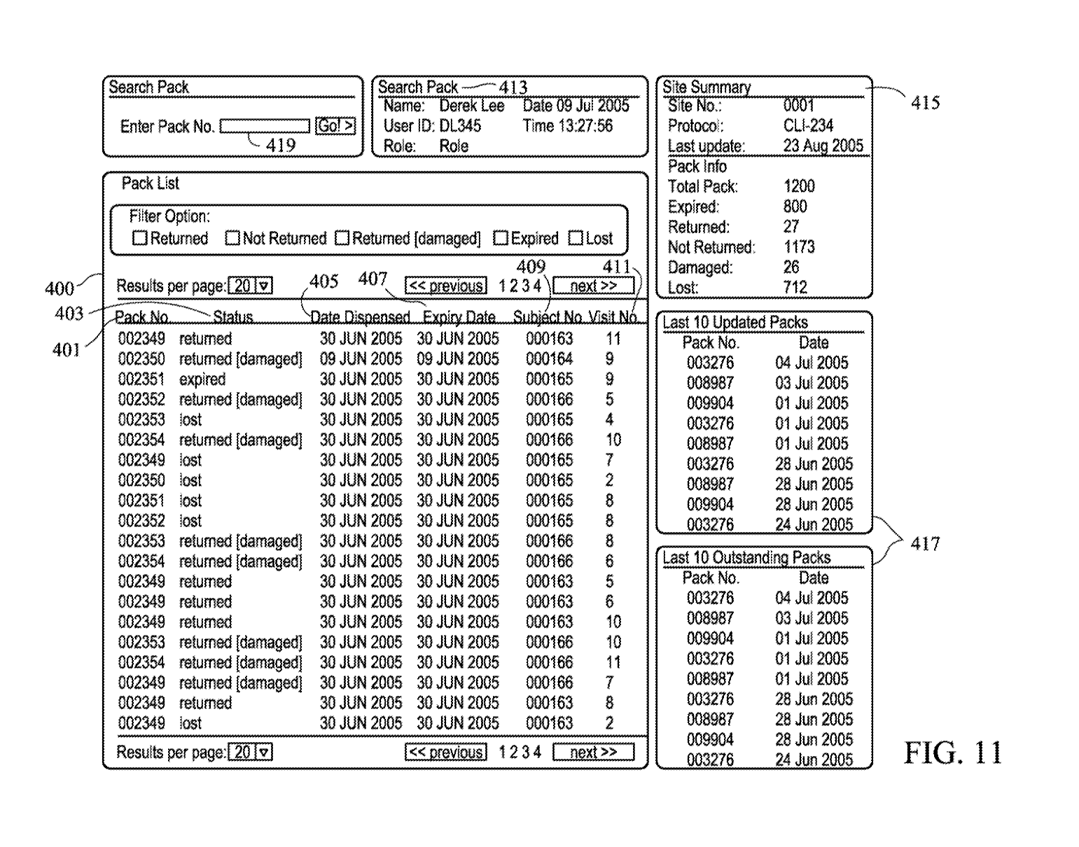

With reference to FIGS. 11 to 14, an embodiment of the present system and methods is illustrated. In this embodiment, the system contains a graphical user interface (GUI) that is displayed on a user terminal. The GUI may be a browswer or other computer implemented software for displaying ARRD data and managing ARRD processes.

With reference to FIG. 11, in one view, a user may be shown pack list data. As seen in FIG. 11, pack list data 400 is displayed to the user and may include data such as pack no. 401, status 403, date dispensed 405, expiry date 407, subject no. 409, a visit no. 411. As further seen in FIG. 11, a variety of data may be displayed to the user including user info data 413, site summary data 415, and user specific or site specific pack data history 417. As described previously, the system may allow a user to search/filter/sort for a particular pack or subject or consignment or site in data field 419, or the user may select a pack directly from the list, which may be a hypertext link or other actuable field.

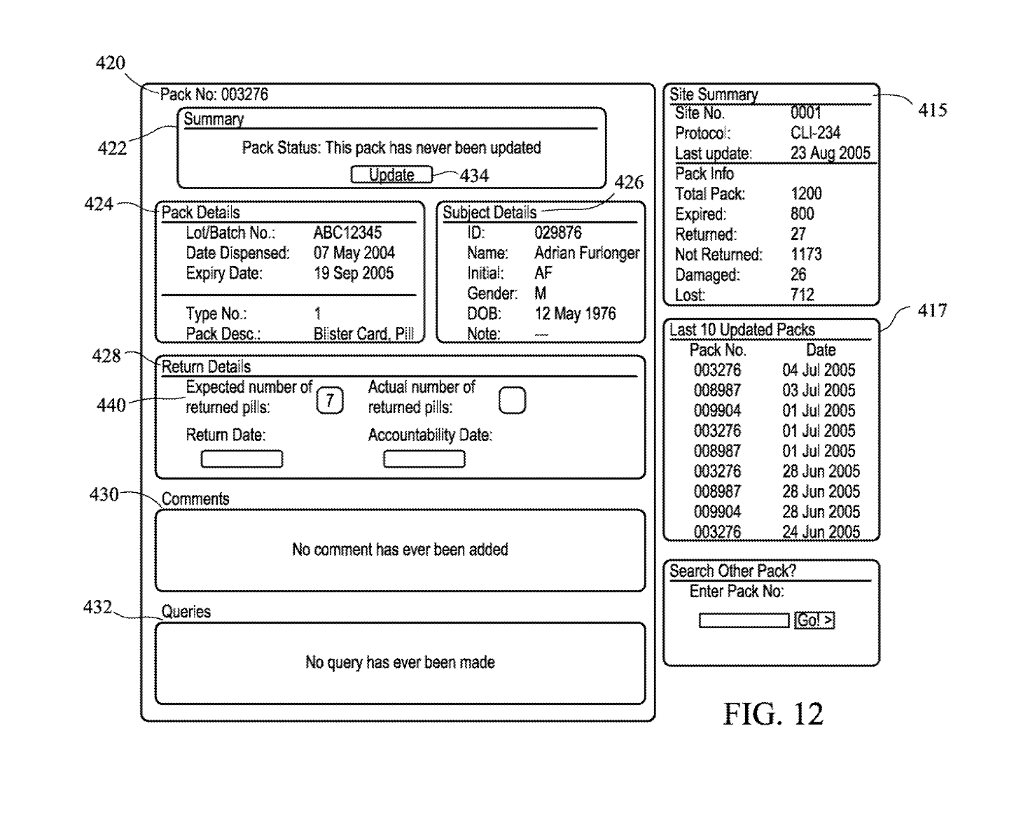

With reference to FIG. 12, a GUI is shown illustrating a screen shot after a user has selected a particular pack. As seen in FIG. 12, the pack no. 420 is displayed to the user. Additionally, the site summary 415 and pack history 417 remains displayed. The user may be provided detailed information concerning a particular pack including pack summary 422, pack details 424, subject details 426, return details 428, and data fields for comments 430 and queries 432. Additionally, the system may provide the user with the ability to update or edit the pack information. Accordingly, as seen in FIG. 12, an update field 434 may be provided to the user.

FIG. 12 also shows another feature of the system in that the system may provide stakeholders with an expected pill count, as seen in expected pill count data field 440. In this embodiment, the system may be configured with a predictive algorithm or rule sets that calculates, based on the study protocol, the expected medication units that should be returned by a particular user during a clinical trial. As one of skill in the art would understand, any number of predictive algorithms may be used depending on the particulars of the study protocol, include expected pill counts, expected return date, dosing regimen, etc. In another embodiment of the system, the system provides the user with data regarding the actual number of medication units returned. In some embodiments, the system may perform rule based or other logic based computations to check the integrity of the data. Accordingly, an expected pill count may be compared to the actual pill count. If a discrepancy exists, an alert or other notification event may be automatically triggered by the system. The alert would notify the appropriate users of the system. In other embodiments, a user may run a report asking for the display of all discrepancies for a particular site. As one of skill in the art would understand, any number of combinations of data checking and notification systems may be used to help manage ARRD activities.

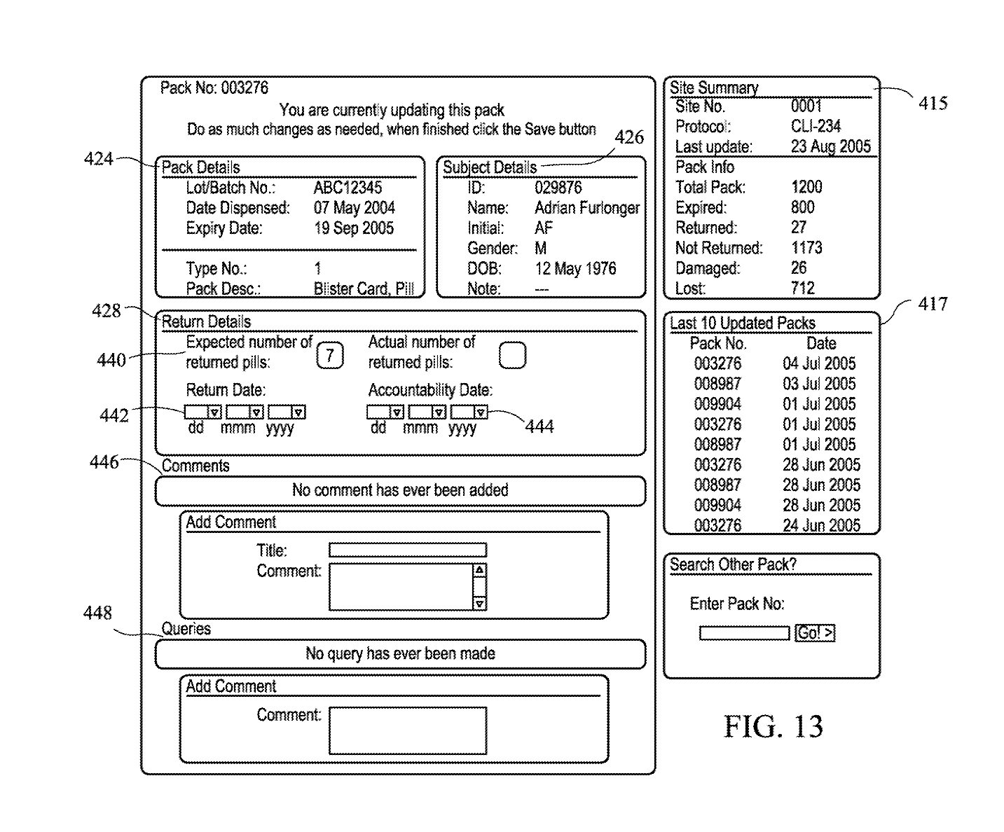

Upon selection of the update field 434, the user may be provided with a variety of data entry fields to edit, enter, or otherwise view relevant pack information. As seen in FIG. 13, the user is continued to be shown pack data including pack details 424, subject details 426, site summary 415 and pack history 417. Furthermore, the user may be able to enter ARRD data such as accountability or return data. Thus, as seen in FIG. 13, return date field 442 may be present to the user for entry of data. Also, accountability field 444 may be provided to the user for entry of data. Additional data field in comment field 446 and query field 448 may be provided to the user.

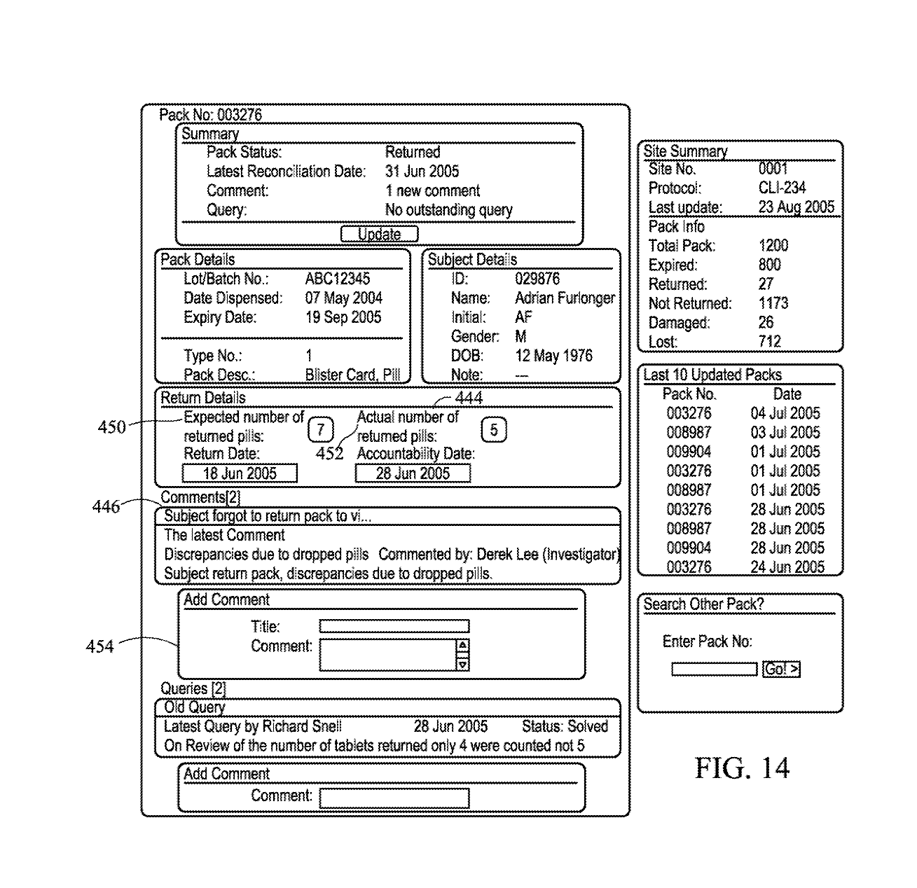

As seen in FIG. 14, the user has entered the actual number of pills returned in accountability field. Because of a discrepancy, a query was conducted to reconcile the expected pill count 450 and actual pill count 452. As seen in query field 454, the reconciliation resulted in the determination that only 4 pills were actually returned. In response to the query, the site user was able to add a comment in comment field 446 explaining the discrepancy between the actual pill count recorded and the actual number of pills returned. In this fashion, multiple users may be alerted to discrepancies in pill count or other ARRD activities and resolve discrepancies from remote locations using a centralized system. Discrepancies can result in hard or soft edit checks. Hard edit checks require the user to adjust out-of-range data, enter a missing value or enter a comment or perform a vital action immediately and must be completed prior to the data on the form being committed to the database. A soft edit check is an advisory message suggesting that data are in conflict or missing etc. but allowing the user to continue and save current data without making further changes or entries or actions.

As one of skill in the art would understand, while mor than one type of stakeholder may have access to the same system and GUI display, in some embodiments not all data is available to all stakeholders. Thus, for example, while the certain patient identifiers may be displayed to site personnel, it may be hidden or not presented to a Sponsor accessing the system.

While various embodiments of the present invention have been described above, it should be understood that they have been presented by way of example only, and not limitation. For example, a variety of programming languages can be used to implement the present invention, such as well-known Java programming language, C++ programming language, C programming language, C# or any combination thereof. Thus, the breadth and scope of the present invention should not be limited by any of the above-described exemplary embodiments, but should be defined only in accordance with the following claims and their equivalents.

It should also be noted that it does not matter where the databases or other data is stored physically. Networks and Internet may connect one data object to a process just as a data bus conects physical memory or non-volatile storage to a processor. Thus, in this discussion and elsewhere, where no particular mention is made of where data is stored, it is assumed not to matter and that a person of ordinary skill could easily make a suitable decision about where to store data—on a vender's server, on a reader, at a home network server, on a third party server, etc. Profile data relating to a particular user may “follow” a user wherever the user goes. Thus, the users privileges and system access remains with the user, wherever they may happen to access the system from. Also note that it has been assumed in the discussions above that some sort of GUI, such as those built into a handled organizer with a touch screen, is associated with the user terminal to allow data to be displayed and entered. The details of the GUI are not important, except as otherwise noted, and could be of any suitable type at the discretion of a designer.

While the invention herein disclosed has been described by means of specific embodiments and applications thereof, numerous modifications and variations can be made thereto by those skilled in the art without departing from the scope of the invention as set forth in the claims.

### Example 2

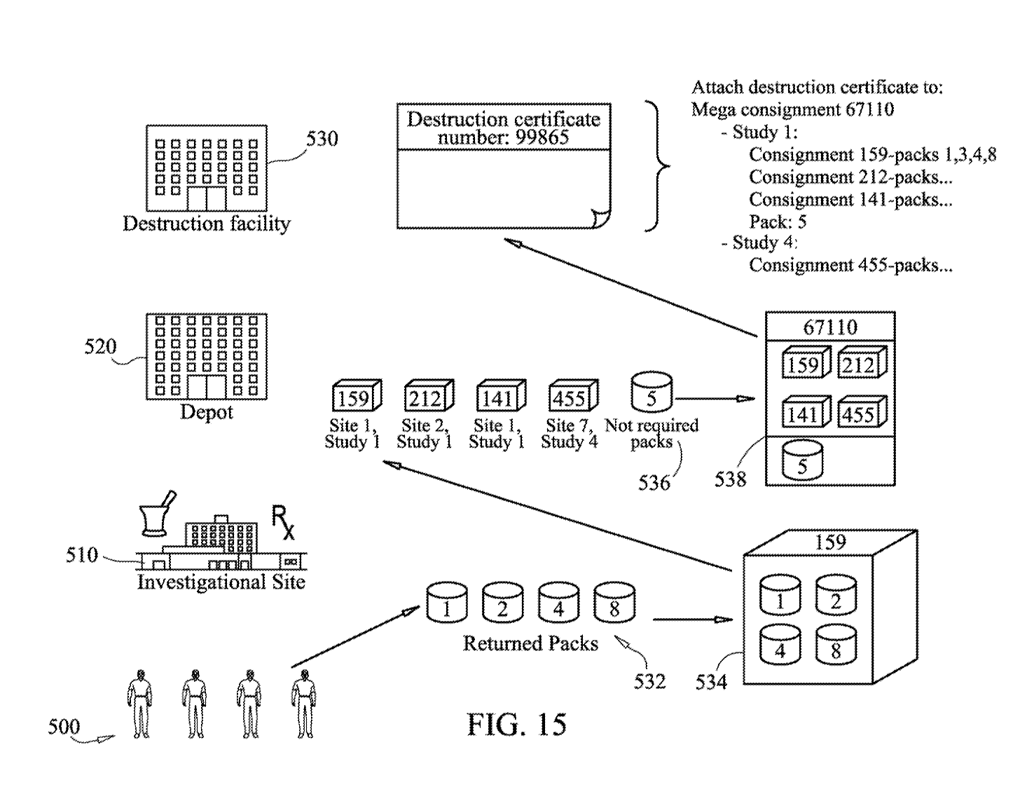

With reference to FIG. 15, another embodiment of the present invention is illustrated. In this embodiment, the system provides one or more users with the ability to track consignments and mega-consignments during destruction processes. As seen in FIG. 15, subjects 500, return packs to an investigational site 510, which in turn may ship consignments and mega-consignments to a depot 520. The depot 520 then ships the packs to a destruction facility 530, which may issue a destruction certificate. The destruction certificate may then be returned to the depot 520 or some other site. Alternatively, a site may destroy a consignment or mega-consignment, in which case they may issue a destruction certificate and enter the activity into the system.

In this illustrative example, the ability of the system to track and display destruction processes is shown. For example, subjects may return uniquely identified packs to an investigational site. As seen in FIG. 15, subjects 500 may return pack nos. 1, 3, 4, and 8 to an investigational site 510. The returned packs 532 may be grouped into a consignment 534, which may be given a unique identifier, which in this example is consignment number 159. Consignment 534 may then be shipped to depot 520. The system of the present invention is capable of storing information regarding the location, status, and composition, e.g. the unique identifier of the packs that make up the consignment, for every consignment tracked by the system. Thus various users of the system are able to enter information at a treatment pack level, consignment level, and mega-consignment level to group treatment packs for destruction processes.

Distribution depot personnel may then be able to record receipt of the consignment and enter and store data related to its role in the destruction processes. For example, depot 520 may receive consignments from more than one site and more than one study. As seen in FIG. 15, in this example depot 520 has received consignments 159, 212, 141, and 455. As seen in FIG. 15, the consignments may be from different sites and correspond to different clinical trials. Additionally, depot 520 may have not required packs 536. Not required packs 536 may include expired treatment packs, damaged packs or excess inventory not used in the study. As seen in FIG. 15, distribution depot 520 may group the various consignments into mega-consignment 538, which is provided a unique identifier 67110. The system provides a distribution depot user or other user with the ability to record, group, and store information related to consignments and mega-consignments. Upon shipment of the mega-consignment 538 to a destruction depot 530, destruction depot 530 may then provide a destruction certificate to distribution depot 520 signifying destruction of the mega-consignment 538. Upon receipt of the destruction certificate, a user may then enter information concerning the destruction, e.g. date, location, etc., of the mega-consignment.

As one of skill in the art would understand, one or more users may be provided with information regarding the location, destruction status, shipment status, composition, etc. of a consignment or mega-consignment. Furthermore, even when viewing data related to a single treatment pack, a user may be provided data with respect to destruction activities related to that single treatment pack. In other uses, users may be provided with a summary of all destruction activity related to one or more clinical trials, one or more investigational sites, and/or one or more distribution depots. In this manner, different stakeholders may be given varying access to destruction activities that help facilitate the management of a clinical study.

We claim:

1. A system for managing drug accountability, the system comprising:
A main database of information concerning drug accountability data for a clinical trial;
A main processor controlling access to said main database;
At least one user processor in communication with said main processor to negotiate access to said main database, said user processor and main processor running a software that provides data concerning drug accountability for a clinical trial to one or more users,
At least one user terminal comprising a user interface that is in communication with said main database, wherein one or more users may enter and view drug accountability data via the at least one user terminal.

2. The system of claim 1, wherein more than one user terminal is in communication with the main database.

3. The system of claim 1, wherein the system is configured to receive, transmit, and store drug accountability data from a destruction facility, depot, site personnel, manufacturing site, Sponsor and monitor.

4. The system of claim 2, wherein each user is assigned user privileges.

5. The system of claim 4, wherein each user privileges may include modification privileges, viewing privileges, access privileges, notification privileges, charting privileges, compilation privileges, extraction privileges, or displaying privileges.

6. The system of claim 1, wherein the user terminal comprises a mobile computing platform.

7. The system of claim 1, wherein a user may enter drug accountability data into the user terminal when the user terminal is offline.

8. The system of claim 7, wherein the system synchronizes the offline drug accountability data entered into the user terminal by the user with the main database when the user terminal is in communication with the main database.

9. The system of claim 3, wherein the system is configured to provide one or more users with data consolidation and reporting functions for submission of data to a regulatory authority.

10. The system of claim 1, wherein said database stores data concerning clinical trial drug accountability data.

11. The system of claim 10, wherein said data is selected from the group consisting of one or more of the following categories: drug dispensation, return process from patient to site, reconciliation process, returns process from site to depot, depot reconciliation, and destruction.

12. The system of claim 1, wherein the system is capable of integrating data from an existing electronic data capture system, clinical trial management system, drug supply management system, or other electronic clinical management system.

13. The system of claim 1, wherein the system is capable of integrating data from an existing trial supply management system.

14. The system under claim 1, wherein the system is capable of operating without integration with other external databases and wherein supply dispatch and ordering data are input into the system.

15. A method comprising:
Enabling an administrator to define a plurality of drug accountability parameters;
Storing the drug accountability parameters in a central database;
Enabling at least a first user to enter drug accountability data corresponding to at least one clinical trial via a wide area network;
Storing said drug accountability data in the central database;
Enabling at least a second user access to said drug accountability data;
Providing the at least first and the at least second users with the ability to view and modify said drug accountability data.

16. The method of claim 15, wherein the plurality of drug accountability parameters include drug dispensation, return process from patient to site, reconciliation process, returns process from site to depot, depot reconciliation, and destruction.

17. The method of claim 15, wherein the drug accountability data includes data related to treatment pack data, dispensation data, consignment data, clinical trial data, user data, site data, return process data, and destruction data.

18. The method of claim 15, wherein the treatment pack data and consignment data is associated with a unique identifier.

19. The method of claim 18, wherein the unique identifier is selected from the group consisting of a barcode, alphanumeric text, and radio frequency tag.

20. The method of claim 15, wherein each user is provided user privileges.

21. The method of claim 20, wherein user privileges provide access to different functionalities of the system.

22. The method of claim 21, wherein at least one of said functionalities includes reporting functionalities.

23. A method of managing drug accountability destruction processes, the method comprising:
a) providing at least one first user with a system for retrieving, viewing, and entering destruction data, the system comprising a user terminal, a database, and a connection between said user terminal and database;
b) storing destruction data regarding a mega-consignment of treatment packs, said data including data related to location, status, and composition of consignments that make up the mega-consignment;
c) providing at least one second user with access to the system for entering, modifying, or viewing data relating to the consignment of treatment packs;
d) storing information entered by said second user in said database;
e) optionally, providing a third user with access to a system for entering destruction data including data related to location, status, and composition regarding a mega-consignment; and
f) providing to one or more users information related to the location, status, composition, and destruction of one or more consignments or mega-consignments.
g) providing to ne or more users reporting of drug accountability status of inventory held at each study site and depot location and providing reporting to enable efficient scheduling of activities to complete drug accountability activities.

24. The method of claim 23, wherein the destruction data relates to more than one clinical study.

25. A system for performing drug accountability activities,
the system comprising at least one user terminal connected to a network, a database for receiving and storing data related to drug accountability activities, and at least one user terminal for entering and viewing drug accountability data into and from the database, wherein the system is configured to associate comments from a first user to said data.

26. The system of claim 25, wherein a second user is shown comments from said first user.

27. The system of claim 26, wherein a second user may respond to comments from said first user, said second user comments being associated with the data related to said first user comments, wherein the second user comments are capable of being displayed to said first user.

28. The system of claim 23, wherein user comments are transmitted to one more recipients automatically.

29. The system of claim 28, wherein said transmission is by email or fax or SMS or outbound automated telephone call.

30. The system of claim 28, wherein said transmission does not include the user comment.

31. The system of claim 30, wherein the notification prompts a user to resolve an issue related to drug accountability activities.

32. The system of claim 28, wherein said notification includes the user comment.

33. The system of claim 32, wherein the notification prompts a user to resolve an issue related to drug accountability activities.
